(19) United States
(12) Patent Application Publication
(10) Pub. No.: US 2026/0158193 A1
(43) Pub. Date: Jun. 11, 2026

(54) MULTIDRUG ANTIMICROBIAL COATING

(71) Applicant: UMC UTRECHT HOLDING B.V., Utrecht (NL)

(72) Inventors: Fatemeh Jahanmard Hosseinabadi, Utrecht (NL); Ahmadreza Majed Hosseinabadi, Utrecht (NL); Saber Amin Yavari, Utrecht (NL)

(21) Appl. No.: 18/706,697

(22) PCT Filed: Nov. 18, 2022
(86) PCT No.: PCT/EP2022/082475

§ 371 Date: May 1, 2024

(30) Foreign Application Priority Data
Nov. 19, 2021 (EP) 21209212.6

(51) Int. Cl. A61L 27/54; A61L 27/34

(52) U.S. Cl. CPC A61L 27/54; A61L 27/34; A61L 2300/404; A61L 2420/02; A61L 2420/06

(57) ABSTRACT

A polymer-based antimicrobial coating, preferably for the prevention and/or treatment of medical-device related infections. The disclosure also relates to a method of preparation of the coating and an intermediate antibiotic formulation thereof for coating a medical device. The coating as part of the current disclosure is compatible with multiple antibiotics, preferably two or more antibiotics with distinct hydrophilicity and/or polarity. The coating as part of the disclosure preferably releases multiple antibiotics in an effective amount for six weeks or more.

### CROSS-REFERENCE TO RELATED APPLICATIONS

This application is a national phase entry under 35 U.S.C. § 371 of International Patent Application PCT/EP2022/082475, filed Nov. 19, 2022, designating the United States of America and published as International Patent Publication WO 2023/089126 A1 on May 25, 2023, which claims the benefit under Article 8 of the Patent Cooperation Treaty of European Patent Application Serial No. 21209212.6, filed Nov. 19, 2021.

### TECHNICAL FIELD

This disclosure relates to a polymer-based antimicrobial coating, preferably for the prevention/and or treatment of medical-device related infections. This disclosure also relates to a method of preparation of the coating and an intermediate antibiotic formulation thereof, which can be applied as a coating on a medical device.

### BACKGROUND

Bacterial infections are a major risk when implanting medical devices. Staphylococcus spp. in particular, can form biofilms on the device surface that become tolerant to treatment. Persisting infections may consequently require removal of the device. For this reason, patients undergoing surgery are often treated prophylactically with antibiotics or other antimicrobial substances to minimize the chance of biofilm formation.

Preferably, a prophylactic treatment provides a sustained release of multiple antibiotics.

First, it has been clinically proven that the delivery of multiple antibiotics is more effective in preventing bacterial (device-related) infections as compared to the use of a single antibiotic. For example, a combination of antibiotics having a different mechanism of action may more optimally eradicate both free-floating bacteria and biofilm-forming bacteria. Second, medical device infections can arise either directly (i.e., immediate infection, via surgical wound contamination) or with weeks to months delay after the surgery (i.e., delayed infection, via spreading of bacteria from a more distant infection site). Therefore, an optimal drug release to protect against early onset and delayed infections would range from hours to weeks depending on the composition.

Prophylactic antibiotics are generally delivered systemically, however local delivery systems for antibiotics can provide higher local drug concentrations, while minimizing systemic drug toxicity. For example, in the orthopedic setting, vancomycin and gentamicin are commonly delivered at the surgical site using polymethyl methacrylate (PMMA) beads as carrier material. However, resorbable polymers offer several advantages over PMMA beads, as they eliminate the need for a second surgery to remove the beads. Moreover, the drug release kinetics can be adjusted to realize appropriate timing of drug release, whereby the drug release is attributed to the drug diffusion and degradation rate, as well as the chemical structure of the chosen polymer.

Antimicrobial polymeric compositions can be applied as a thin film (also referred to as “antimicrobial coating”) on the device surface by means of different technologies, to optimally protect the implant surface from bacterial colonization. The process for manufacturing an antimicrobial polymeric coating generally involves the following steps:
1. dissolving a polymer in a solvent to obtain a polymeric formulation;
2. dissolving an antibiotic in a solvent to obtain an antibiotic formulation;
3. mixing the polymeric formulation with the antibiotic formulation to obtain a polymer-antibiotic formulation;
3. applying the polymer-antibiotic formulation as a coating on a medical device;
4. (optionally) evaporating the solvents.

Despite the great clinical need, the aforementioned process does not easily allow for the incorporation of multiple antibiotics into the same polymeric coating. The manufacture of multidrug polymeric coatings is particularly challenging when the two or more antibiotics (and the necessary solvents) have different physicochemical properties (hydrophilicity, polarity, and/or water-miscibility), such as for the following reasons:
First, antibiotics having different hydrophilicity/polarity are thought to require different organic solvents. Hydrophilic drugs suffer from low loading efficiency in hydrophobic polymer compositions. Hence, lipophilic drugs are often preferred in hydrophobic polymeric compositions and hydrophilic drugs are often preferred in hydrophilic polymeric compositions. In addition, lipophilic drugs are soluble in a wide range of organic solvents, whereas hydrophilic drugs are only compatible with a limited number of organic solvents (US2010215716A). The simple mixing of antibiotics in the respective organic solvents typically leads to low drug solubility, poor miscibility, and/or drug precipitation. Ultimately, this leads to poor drug loading efficiency and unfavorable drug release profiles (i.e., insufficient drugs or no sustained release).
Second, providing a high polymer content in a polymeric formulation typically leads to high viscosity of the polymer-antibiotic formulation. This hampers the formation of a homogenous and thin film coating by one of the various coating methods. For example, electrospraying is a preferred coating method, but requires a relatively low viscosity. A high viscosity of a polymer-antibiotic coating may result in inhomogeneous or non-uniform coating, and also poor drug release profiles. Having a higher polymer content in the polymeric formulation may also allow higher amounts of (two or more) antibiotics to be incorporated, since a high polymer-drug ratio can be ensured. The higher amounts of (two or more) antibiotics may provide better encapsulation, improved coating, and a prolonged release of drugs over time.
Third, drug release rates from polymer compositions are influenced by the drug diffusion rates. It is generally believed that incorporating a hydrophilic (polar) and a hydrophobic (nonpolar) antibiotic in a polymeric coating leads to unpredictable diffusion rates of the antibiotics. Moreover, it is often observed in the prior art that a mismatch in the diffusion rates of hydrophilic (polar) and hydrophobic (nonpolar) antibiotics occurs if not applied adequately in the same polymer.

Several approaches have been investigated to co-deliver multiple, physicochemically-distinct antibiotics. An example is the use of lipid nanoparticles such as liposomes to carry lipophilic antibiotics in the phospholipid double layer while hydrophilic drugs can be encapsulated into the aqueous core (Forier et al. J Control Release. 2014 Sep. 28; 190:607-23). However, this methodology does not provide a sufficiently sustained drug release profile to protect against both early onset (hours) and delayed infections (weeks). Alternatively, the co-delivery of drugs with different physicochemical properties (such as hydrophilicity, polarity, and/or water-miscibility) has opted for the use of different polymeric compositions to tune the individual drug release behavior in a wide timespan (Ritsema. Int J Pharm 2018 September 11; 548 (2): 730-739, Jahanmard et al. J Control Release. 2020 Oct. 10; 326:38-52). For example, hydrophilic and hydrophobic antibiotics have been encapsulated into different polymers, which are then combined in a certain architecture. Using this approach, hydrophilic vancomycin has been encapsulated into hydrophobic polycaprolactone (PCL) polymer fibers, and hydrophobic rifampin has been encapsulated into hydrophobic poly(lactic glycolic acid) PLGA fibers, whereby the fibers were subsequently applied in a layered fashion (Jahanmard et al. J Control Release. 2020 Oct. 10; 326:38-52.).

There remains a need for multidrug antimicrobial polymeric coatings, foremost coating formulations that are easily adaptable to incorporate different antibiotics of choice (i.e., depending on the clinical scenario) within a single thin film and/or within a single polymer matrix.

More specifically, there is a need for:
antimicrobial coatings, foremost coating formulations, which incorporate two or more antibiotics with different physicochemical properties, such as different hydrophilicity (polarity) in a single polymer matrix and preferably in a single layer coating within the same polymer matrix.
antimicrobial formulations, which incorporate two or more antibiotics with different physicochemical properties, such as different hydrophilicity (polarity), with appropriate formulation characteristics (low viscosity, high drug load, high drug solubility) in order to be applied for coating means (preferably spraying such as electrospraying).
antimicrobial coatings, which encapsulate two or more antibiotics (e.g., with different physicochemical properties) in sufficient high amounts in a single polymer matrix, e.g., to provide drug release profiles that extend at least several weeks (preferably six weeks or longer) after implantation in an amount effective to inhibit growth and/or kill microorganisms.

### BRIEF SUMMARY

The current disclosure relates to an antimicrobial formulation (a coating solution), preferably a multidrug antimicrobial formulation, and a preparation method thereof. The disclosure furthermore relates to an antimicrobial coating obtained when applying the antimicrobial formulation as a thin film on a medical device, such as by means of a spraying (e.g., electrospraying) spin coating, or electrospinning. The current disclosure also relates to the use of the antimicrobial formulation and the antimicrobial coating in the prevention and/or treatment of medical device-related infections, particularly orthopedic implant-related infections.

It was found that, in a process for manufacturing a polymer-antibiotic formulation for subsequent use in applying an antimicrobial coating, the viscosity of the formulation can be reduced dramatically by using a hydrophobic (non-polar) antibiotic such as a rifamycin, a macrolide, or a tetracylin. It was found that the hydrophobic (non-polar) antibiotic is preferably applied in the following way to achieve a formulation with a reduced viscosity:
a) providing a first solvent with a polymer, wherein the first solvent is a fluorinated solvent and the polymer is an aliphatic polyester;
b) providing a second solvent with a first antibiotic, wherein the first antibiotic is hydrophobic (non-polar) and/or is soluble in a non-polar organic solvent;
c) mixing the first solvent with the polymer of step a) and the second solvent with the first antibiotic of step b) to obtain a mixture that has a lower dynamic viscosity than the first solvent with the polymer of step a);
d) providing a third solvent with a second antibiotic, wherein the second antibiotic is preferably hydrophilic (polar) and/or soluble in a polar aprotic organic solvent;
e) mixing the mixture obtained in step c) and the third solvent with the second antibiotic of step d) to obtain the antimicrobial formulation.

The first antibiotic and the secondary antibiotic may be selected based on a specific clinical need (e.g., type of infection, type of surgery, type of medical device etc.).

The antimicrobial formulation according to the current disclosure preferably has a dynamic viscosity of 1 mPa·s-20 Pas (pascal-second), preferably 1 mPa·s-2 Pa·s, more preferably 1 mPa·s-200 mPa·s. Lower viscosities may particularly allow coating by spraying, including electrospraying (e.g., at dynamic viscosity of 1 mPa·s-200 mPa·s) or by spinning, including spin coating and electrospinning (e.g., at dynamic viscosity of 200 mPa·s-20 Pa·s).

In a preferred embodiment, the antimicrobial formulation according to the current disclosure has a dynamic viscosity of 0.1-500 mPa·s, preferably 1-250 mPa·s, more preferably 10-200 mPa·s, even more preferably 20-100 mPa·s.

Preferably, the antimicrobial formulation according to the current disclosure has a high polymer and drug content. For example, the polymer content may be 40-90%, preferably 50-80%, by weight of the antimicrobial formulation. For example, a first antibiotic (preferably a hydrophobic antibiotic such as rifampicin) and a second antibiotic (preferably a hydrophilic antibiotic such as vancomycin) may be independently present in an amount of 5-40%, preferably 10-30%, by weight of the antimicrobial formulation. In addition or alternatively, the polymer may be present (w//v) at 10-300 mg/ml of the antimicrobial formulation, preferably 50-150 mg/ml. The first antibiotic (preferably a hydrophobic antibiotic such as rifampicin) and a second antibiotic (preferably a hydrophilic antibiotic such as vancomycin) may be independently present at a concentration of 10-200 mg/ml of the antimicrobial formulation, preferably 20-100 mg/ml.

In an embodiment, the antimicrobial formulation is prepared by providing a hydrophobic (non-polar) antibiotic in a non-polar or a polar aprotic solvent. To lower the viscosity of a polymer-antibiotic formulation, the antibiotic solution is mixed with a polymer (preferably an aliphatic polyester, more preferably a hydrophobic aliphatic polyester) provided in a fluorinated solvent (preferably a fluorocarbon or a fluoroalcohol). This polymer-antibiotic formulation may be effectively combined with a solution comprising a second antibiotic that is provided to yield a multidrug antimicrobial formulation. For example: PDLG (DL-lactide/glycolide copolymer) may be provided in tetrafluoroethylene as a polymer solution, rifampicin may be provided in a non-polar organic solvent (preferably chloroform) as a first antibiotic solution, and vancomycin may be provided in a polar aprotic solvent (preferably dimethyl sulfoxide) as a second antibiotic solution. Following a coating step, this may yield an antimicrobial coating comprising a high content of a hydrophobic (non-polar) and a hydrophilic (polar) antibiotic both dispersed in a single polymer matrix.

It was found that the antimicrobial formulation according to the current disclosure offers several advantages in terms of coating manufacturing, among other possible advantages. The antimicrobial formulation allows coating by techniques such as spraying or spinning. The antimicrobial formulation according to the current disclosure is capable of incorporating a high drug content. In addition, the reduced viscosity of the antimicrobial formulation if the current disclosure may lead to a more uniform coating, faster drying times, less shrinkage, more complete fill, and fewer air pockets, among others.

It was furthermore found that the antimicrobial formulation according to the current disclosure and the subsequent application as a coating is easily adaptable to (multiple) antibiotics of choice, even if the desirable antibiotics have distinct physicochemical properties (e.g., hydrophilicity, polarity). For example, it was shown that a hydrophilic aminoglycoside (preferably tobramycin, gentamicin, streptomycin, or neomycin) or a hydrophilic glycopeptide (preferably vancomycin) may be combined with a hydrophobic rifamycin (preferably rifampin), a hydrophobic macrolide (preferably erythromycin), or a hydrophobic tetracyclin (preferably minocycline) while maintaining high solubility of the drugs and low viscosity of the antimicrobial formulation.

Due to the advantages provided by the antimicrobial formulation according to the present disclosure, a multidrug antimicrobial coating with a high polymer and/or high drug content can be achieved. For example, the polymer may be present in an amount of 40-90%, preferably 50-80%, by weight of the antimicrobial coating. The first and the second antibiotic may be independently present in an amount of 5-40%, preferably 10-30%, by weight of the antimicrobial coating.

It was found that the high drug content (and use of combinatorial antibiotics) in the antimicrobial coating results in long-term delivery of drugs, e.g., at least six weeks. Moreover, a sustained antimicrobial effect against Staphylococcus spp (i.e., the most common cause of metal implant infections) was found, e.g., killing of Staphylococci spp up to six weeks was demonstrated.

As a preferred embodiment of the current disclosure, it was demonstrated that the coating is suitable for the long-term delivery of rifampin, together with a secondary (hydrophilic or hydrophobic) antibiotic. Rifampin, uniquely, can kill Staphylococci spp. in both planktonic or biofilm state, however it must be co-delivered with a secondary antibiotic to prevent bacterial resistance against rifampin. Rifampin is a large hydrophobic molecule. Secondary antibiotics can be common used broad-spectrum antibiotics such as gentamicin, vancomycin or tobramycin, however these typically are large hydrophilic/polar drugs. It was found that such preferred drug combinations can be incorporated into a single polymer and as a single thin film coating by means of the antimicrobial formulation of the current disclosure.

In a more preferred embodiment, the antimicrobial coating as part of the disclosure is suitable for the long-term delivery of rifampin together with vancomycin. Unexpectedly, it was found that the presence of the hydrophilic antibiotic (e.g., vancomycin) may delay the diffusion of the hydrophobic antibiotic (e.g., rifampin) from the polymer matrix. Vice versa, the presence of rifampin may delay the diffusion of vancomycin. As both antibiotics are released more simultaneously rather than more, the antimicrobial effect is enhanced and the chance of bacterial resistance developing is reduced. It is considered that a hydrophobic antibiotic does not easily diffuse out of hydrophobic polymeric matrices and that incorporating a hydrophilic antibiotic can help for better release and diffusion of the hydrophobic antibiotic.

### BRIEF DESCRIPTION OF THE DRAWINGS

FIG. 1. Rifampicin release profile from coated titanium discs. The role of a hydrophobic drug and/or a non-polar organic solvent was tested. PDLG (DL-lactide/glycolide copolymer) was used as the polymer in the coating solution (dissolved in tetrafluoroethylene). Values represent the mean+/−standard deviation (n=3). Rif: Rifampicin, Van: vancomycin, DMSO: dimethyl sulfoxide.

FIG. 2. Vancomycin release profile from coated titanium discs. The role of a hydrophobic drug and/or a non-polar organic solvent was tested. PDLG (DL-lactide/glycolide copolymer) was used as the polymer in the coating solution (dissolved in tetrafluoroethylene). Values represent the mean+/−standard deviation (n=3). Rif: Rifampicin, Van: vancomycin, DMSO: dimethyl sulfoxide.

FIG. 3. Rifampicin release profile from coated titanium discs. Different hydrophobic/hydrophilic drug combinations were tested. PDLG (DL-lactide/glycolide copolymer) was used as the polymer in the coating solution (dissolved in tetrafluoroethylene). Values represent the mean+/−standard deviation (n=3). Rif: Rifampicin, DMSO: dimethyl sulfoxide.

FIG. 4. Vancomycin release profile from coated titanium discs. Different hydrophobic/hydrophilic drug combinations were tested. PDLG (DL-lactide/glycolide copolymer) was used as the polymer in the coating solution (dissolved in tetrafluoroethylene). Values represent the mean+/−standard deviation (n=3). Rif: Rifampicin, Van: Vancomycin, DMSO: dimethyl sulfoxide.

FIG. 5. Rifampicin release profile from coated titanium discs. Different polymers (solution A) were compared (dissolved in tetrafluoroethylene). The coating solutions further comprised Rifampicin/chloroform (Solution A) and Vancomycin/dimethyl sulfoxide (Solution D). In addition, different coating methods were tested. Values represent the mean+/−standard deviation (n=3). PLDG: DL-lactide/glycolide copolymer, PLDL: L-lactide/DL-lactide copolymer, PLC: L-lactide/caprolactone copolymer, PL: Poly(L-lactide), PCL: Polycaprolactone.

FIG. 6. Vancomycin release profile from coated titanium discs. Different polymers (solution A) were compared (dissolved in tetrafluoroethylene). The coating solutions further comprised Rifampicin/chloroform (Solution A) and Vancomycin/dimethyl sulfoxide (Solution D). In addition, different coating methods were tested. Values represent the mean+/−standard deviation (n=3). PLDG: DL-lactide/glycolide copolymer, PLDL: L-lactide/DL-lactide copolymer, PLC: L-lactide/caprolactone copolymer, PL: Poly(L-lactide), PCL: Polycaprolactone.

FIG. 7. Rifampicin release profile from coated titanium discs. PDLG (DL-lactide/glycolide copolymer) was used as the polymer in the coating solution (dissolved to 200 mg/ml in tetrafluoroethylene). The coating solution further comprised Rifampicin/chloroform (Solution A, 70 mg/ml or 50 mg/ml) and Vancomycin/dimethyl sulfoxide (Solution D, 70 mg/ml or 50 mg/ml). Values represent the mean+/−standard deviation (n=3). PDLG=DL−lactide/glycolide copolymer, Rif: Rifampicin, Van: vancomycin.

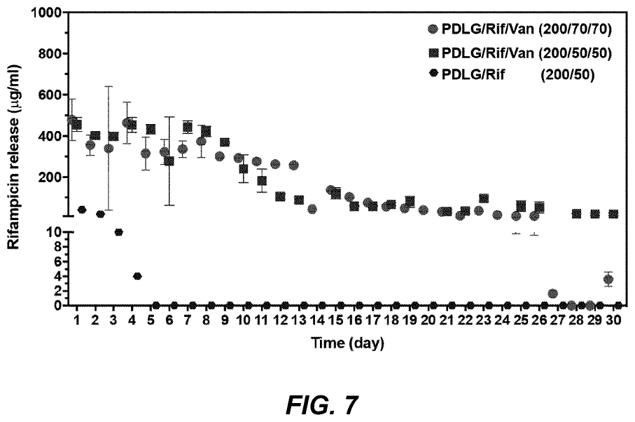

FIG. 8. Vancomycin release profile from coated titanium discs. PDLG (DL−lactide/glycolide copolymer) was used as the polymer in the coating solution (dissolved to 200 mg/ml in tetrafluoroethylene). The coating solution further comprised Rifampicin/chloroform (Solution A, 70 mg/ml or 50 mg/ml) and Vancomycin/dimethyl sulfoxide (Solution D, 70 mg/ml or 50 mg/ml). Values represent the mean+/−standard deviation (n=3). PDLG=DL−lactide/glycolide copolymer, Rif: Rifampicin, Van: vancomycin.

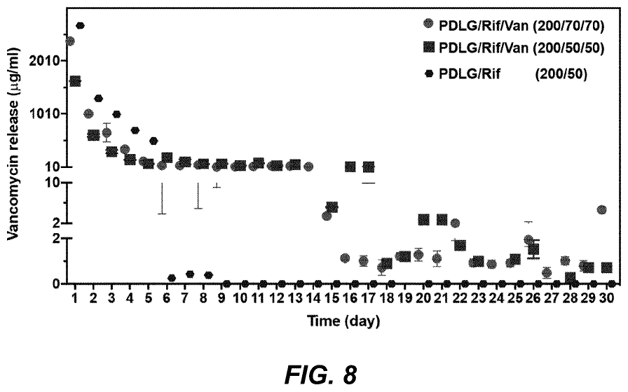

FIG. 9. Differences in viscosity of coating solutions obtained by two methods. In Method A, rifampicin was first dissolved in chloroform and subsequently mixed with a polymeric (PLGA) solution. In Method B, the rifampicin was directly added to a polymeric (PCL) solution. The rifampicin/polymeric solutions were mixed with a vancomycin solution to obtain the coating solution. The solutions according to Method A and Method B were placed on a shaker and the speed of the shaker was increased from 0 to 500 rpm and then decreased until a complete stop. The images were taken every second for 45 seconds. The horizontal lines indicate the flow of the solutions during the shaking process.

FIG. 10. AFM images at different magnifications of a coating obtained by electrospraying of a coating solution according to Method A.

FIG. 11. AFM images at different magnifications of a coating obtained by electrospraying of a coating solution according to Method B.

### DETAILED DESCRIPTION

The present disclosure relates to a method for preparing an antimicrobial formulation, the method comprising:
a) providing a first solvent with a polymer, wherein the first solvent is preferably a fluorinated solvent and the polymer is preferably an aliphatic polyester;
b) providing a second solvent with a first antibiotic, wherein the first antibiotic is preferably hydrophobic and/or soluble in a non-polar organic solvent;
c) mixing the first solvent with the polymer of step a) and the second solvent with the first antibiotic of step b) to obtain a mixture that preferably has a lower dynamic viscosity than the first solvent with the polymer of step a);
d) providing a third solvent with a second antibiotic, wherein the second antibiotic is preferably hydrophilic and/or soluble in a polar aprotic organic solvent;
e) mixing the mixture obtained in step c) and the third solvent with the second antibiotic of step d) to obtain the antimicrobial formulation.

In accordance, with the foregoing, the present disclosure relates to an antimicrobial formulation, which can be obtained by the method for preparing an antimicrobial formulation as disclosed herein, the antimicrobial formulation comprising:
a polymer, preferably at a concentration of 10-300 mg/ml of the antimicrobial formulation, more preferably 50-150 mg/ml, wherein the polymer is preferably an aliphatic polyester, such as a hydrophobic aliphatic polyester, more preferably one or more of PDLG (DL-lactide/glycolide copolymer), PLGA (L-lactide/glycolide copolymer), PLC (L-lactide/caprolactone copolymer), PCL polycaprolactone, PD [poly(D-lactide)], PL [poly(L-lactide)], or PDL [(poly(DL-lactide)], most preferably PDLG;
a first antibiotic, preferably at a concentration of 10-200 mg/ml of the antimicrobial formulation, more preferably 20-100 mg/ml, wherein the first antibiotic is preferably hydrophobic and/or soluble in a non-polar organic solvent, preferably one or more of a rifamycin, a hydrophobic hydroquinolone, a macrolide, a tetracycline, or a lincosamide, more preferably rifampin;
a second antibiotic, preferably at a concentration of 10-200 mg/ml of the antimicrobial formulation, more preferably 20-100 mg/ml, wherein the second antibiotic is preferably hydrophilic and/or soluble in a polar aprotic organic solvent, preferably one or more of a glycopeptide, an aminoglycoside, a beta-lactam, a carbapenem, a hydrophilic fluoroquinolone, or a streptogramin, more preferably vancomycin;
a non-polar organic solvent, preferably at a concentration of 10-50% (v/v) with respect to the antimicrobial formulation, more preferably 20-40% (v/v), wherein the non-polar organic solvent is preferably one or more of chloroform, pentane, hexane, benzene, diethyl ether, and 1,40-Dioxane, more preferably chloroform; and/or
a polar aprotic organic solvent, preferably at a concentration of 1-40% (v/v) with respect to the antimicrobial formulation, more preferably 10-30% (v/v), wherein the polar aprotic solvent is preferably one or more of dimethyl sulfoxide, dichloromethane, tetrahydrofuran, ethyl acetate, acetonitrile, dimethylformamide, dimethyl sulfoxide, acetone, and hexamethylphosphoric triamde, more preferably dimethyl sulfoxide.

The current disclosure also relates to a method for preparing an antimicrobial coating, the method comprising applying the antimicrobial formulation disclosed herein on a substrate (preferably a medical device, more preferably an orthopedic implant) and removing the one or more solvents in the antimicrobial formulation, wherein the applying is preferably performed by dip coating, spin coating, spraying, electrospinning, electrophoretic deposition, sputter coating, thermal spraying, plasma spraying, sol-gel, layer-by-layer coating, more preferably electrospraying.

The current disclosure furthermore relates to an antimicrobial coating, which can be obtained by the method for preparing an antimicrobial coating as disclosed herein, the antimicrobial coating comprises:
a polymer, preferably in an amount of 40-90%, more preferably 50-80%, by weight of the antimicrobial coating, wherein the polymer is an aliphatic polyester, preferably a hydrophobic aliphatic polyester;
a first antibiotic, preferably in an amount of 5-40%, more preferably 10-30%, by weight of the antimicrobial coating, wherein the first antibiotic is hydrophobic and/or soluble in a non-polar organic solvent; and
a second antibiotic, preferably in an amount of 5-40%, more preferably 10-30%, by weight of the antimicrobial coating, wherein the second antibiotic is hydrophilic and/or soluble in a polar aprotic organic solvent,
wherein the first antibiotic and the second antibiotic are comprised in a matrix effected by the polymer.

In an embodiment, the method for preparing an antimicrobial formulation as disclosed herein comprises:
a) providing a first solvent with a polymer, wherein the first solvent is a fluorinated solvent and the polymer is an aliphatic polyester;
b) providing a second solvent with a first antibiotic, wherein the first antibiotic is hydrophobic and/or soluble in a non-polar organic solvent;
c) mixing the first solvent with the polymer of step a) and the second solvent with the first antibiotic of step b) to obtain a mixture that has a lower dynamic viscosity than the first solvent with the polymer of step a);
d) providing a third solvent with a second antibiotic, wherein the second antibiotic is hydrophilic and/or soluble in a polar aprotic organic solvent;
e) mixing the mixture obtained in step c) and the third solvent with the second antibiotic of step d) to obtain the antimicrobial formulation.

The term “antimicrobial” as used herein relates to a substance that inhibits growth and/or kills microbes (e.g., bacteria, fungi, virus, preferably bacteria). The term “antimicrobial” may also be used to describe the growth-inhibiting and/or killing effect of an antimicrobial substance on microbes. Antimicrobials as disclosed herein preferably include one or more of antibiotics, antivirals, antifungals and antiparasitics. Preferably, the antimicrobial as disclosed herein is not an antiseptic.

The term “antibiotic” as used herein relates to an antimicrobial compound or drug that targets bacteria and/or fungi; and/or disrupts functioning of bacteria and/or fungi. An antibiotic is typically an organic substance of microbial origin and generally binds to a specific molecular target in bacteria and fungi. Their mechanism of action may be one or more of: inhibition of nucleic acid synthesis, inhibition of biosynthesis of proteins, disruption of membranes, functioning as antimetabolites, inhibition of cell wall biosynthesis. In a preferred embodiment, the coating and/or coating solution as part of the current disclosure does not comprise an antiseptic. Antiseptics relate to chemicals that typically reduce bacterial growth or kill bacteria by non-specific mechanisms. Antiseptics are typically classified according to their mechanism of action as small molecules that indiscriminately react with organic compounds and kill microorganisms (peroxides, iodine, phenols), or more complex molecules that disrupt the cell walls of the bacteria. Common examples of antiseptics are phenols, alcohol, quinolines, diguanides (e.g., chlorhexidine), alcohols, peroxides, and iodine. Antiseptics are commonly applied topically, and usually cannot be used internally or ingested due to their toxicity. Therefore, antiseptics are generally not suitable as antimicrobial agent or antibiotic as taught herein.

In an embodiment, the current disclosure does not use an antiseptic such as an antiseptic disclosed above.

The term “polymer” as used herein may refer to either/both a homopolymer and a copolymer. A homopolymer is a polymer comprising only one monomer. A copolymer is a polymer formed when two (or more) different types of monomers are linked in the same polymer chain. The copolymer herein may be an alternating copolymers (typically comprising only two types of repeat units, which are arranged alternative along the polymer chain), a block copolymer (typically comprising repeat units that exist in blocks of the same), or a graft copolymer (typically comprising branches of different chemical structures attached to a main chain). The polymer as disclosed herein may be a natural polymer or and/or a synthetic polymer.

The polymer as disclosed herein may be one or more of the following, or a copolymer comprising one or more of the following: a poly(α-ester), polyglycolide, polylactide [e.g., poly(L-lactic acid) (PLLA), poly(D-lactic acid) (PDLA), poly(D,L-lactic acid) (PDLLA), meso-poly(lactic acid)], polycaprolactone, poly(propylene fumarate), a polyanhydride, a polyacetal, a poly(ortho ester), a polycarbonate, a polyurethane, a polyphosphazene, a polyphosphoester. The polymer as disclosed herein may be a combination polymer, i.e., polymers in which monomers contain multiple degradable groups [e.g., a poly(ester ether), a poly(amide ester)]. The polymer as disclosed herein may be a protein such as one or more of collagen, elastin, albumin, and fibrin. The polymer as disclosed herein may be a polysaccharide (e.g., hyaluronic acid, chondroitin sulfate, chitin, chitosan, alginate, dextran, agarose, mannan, inulin). The polymer as disclosed herein may be a natural poly(amino acid) [e.g., poly(γ-glutamic acid), poly(L-lysine)]. The polymer as disclosed herein may be a synthetic poly(amino acid) [e.g., poly(L-glutamic acid), poly(aspartic acid)].

Preferably, the polymer as taught herein is resorbable. The term “resorbable” as used herein is used to describe a material that degrades in an aqueous medium and/or after implantation into the body over time. The resorption of a material (e.g., coating) is generally determined by a combination of physico-chemical degradation, enzymatic activity, and cellular degradation (immune response, foreign body response). Preferably, the rate of resorption is established in a controlled in vitro environment, even though the rate of resorption of a material may be different inside of the body. A controlled in vitro environment may comprise that the material is incubated in an excess of PBS (pH 7.4) at 37° C. and wherein the polymer is “resorbable” if at least 50 wt. % is degraded after 8 weeks, preferably after 6 weeks, more preferably after 4 weeks.

The term “resorbable” as sued herein may be used interchangeably with “bioresorbable,” “(bio) degradable,” and “(bio) erodible.”

The term “solvent” as used herein relates to a liquid, in which materials/substances can dissolve to form a solution. Preferably, a liquid is said to act as solvent for a certain material/substance, if the material/substance has a solubility of at least 0.1 in 100 (0.1 mg/ml), preferably 1 in 100 (1 mg/ml), in the liquid.

The term “soluble” as used herein means that the substance of interest (e.g., polymer, or antibiotic) has a solubility of at least 0.1 in 100 (0.1 mg/ml), preferably 1 in 100 (1 mg/ml), in a liquid (preferably a solvent). Solubility is typically measured according to techniques wherein an excess amount of substance is suspended in a liquid (solvent) and shaken at 25° C. for 24 hours. After the solution is filtered, the concentration of the substance in solution is measured e.g., using UV (ultraviolet) or HPLC (High-performance liquid chromatography).

The term “fluorinated solvent” as used herein refers to a solvent comprising fluorine.

The term “fluorinated solvent” may be herein used interchangeably with “fluorous solvent” and “fluorosolvent.” The term “fluorinated solvent” as disclosed herein preferably is one or more of or a fluoroalcohol or a fluorocarbon. The fluoroalcohol as disclosed herein may be one or more of (2,2,2,-trifluoroethanol), trifluoroethyl alcohol, hexafluoro-2-propanol, and trifluoropropanol. The fluorocarbon as disclosed herein may be one or more of dichlorofluoromethane (CHCl₂F), trichlorofluoromethane (CCl₃F), tetrafluoromethane (CF₄), difluorodichloromethane (CHCl₂F₂), chlorodifluoromethane (CHClF₂), and tetrafluoroethylene (TFE, C₂F₄).

In various embodiments, the solvent for the polymer as disclosed herein is not a fluorinated solvent, such as disclosed herein. In an embodiment, the solvent for the polymer as disclosed herein is a non-polar solvent. In an embodiment, the solvent for the polymer as disclosed herein is a polar aprotic solvent. In an embodiment, the solvent for the polymer as disclosed herein is a polar protic solvent.

Herein, the term “hydrophobic” may be used interchangeably with the term “non-polar” or “lipophilic,” preferably when describing an antibiotic or a polymer. Herein, the term hydrophilic may be used interchangeably with “polar,” preferably when describing an antibiotic or a polymer. A hydrophobic and/or non-polar compound (e.g., antibiotic or polymer) as disclosed herein preferably has a Log P value greater than 0 and/or a P value greater than >1. A hydrophilic and/or polar compound (e.g., antibiotic or polymer) as disclosed herein preferably has a Log P value of less than 0 and/or a P value of less than 1. The “Log P” as used herein preferably refers to the n-octanol-water Partition coefficient as a measure of how hydrophilic (polar) or hydrophobic (non-polar, lipophilic) a molecule/compound is. It indicates how readily a molecule/compound will partition between an aqueous and organic phase. A more polar, hydrophilic molecule/compound will have a lower log P and prefer to reside in the aqueous phase. A more non-polar, hydrophobic molecule/compound will have a higher log P, and will partition preferably into an organic phase. The log P equals log 10(Partition coefficient), wherein the Partition coefficient (P) equals the concentration of solute in the organic partition, divided by the concentration of solute in the aqueous partition. For example, a Log P value of 1 means there is a 10:1 partitioning in organic:aqueous phases. The person skilled in the art is well aware of the most accurate method to determine the Partition coefficient (n-octanol-water) for a given molecule/compound. Typically, a suitable method is the shake flask method (OECD. 107: Partition Coefficient (n-octanol/water):Shake Flask Method. OECD Guidelines for the Testing of Chemicals, 1995).

The “non-polar solvent” as used herein preferably refers to a solvent with a dipole moment of less than 2.0 (debye, D), preferably less than 1.5, a relative permittivity of less than 15.0, preferably less than 5.0, and/or to a solvent that is immiscible with water. The non-polar solvent as disclosed herein is preferably one or more of chloroform, pentane, hexane, benzene, diethyl ether, and 1,40-Dioxane, more carbon tetrachloride, and methylene chloride.

The “polar protic solvent” as used herein preferably refers to a solvent with a dipole moment of 1.5-2.0 (debye, D), a relative permittivity of more than 15.0, preferably more than 30.0, and/or to a solvent that is miscible with water. The polar protic solvent as disclosed herein is preferably one or more of water, methanol, ethanol, isopropyl alcohol, n-propanol, n-butanol, and acetic acid.

The “polar aprotic solvent” as used herein preferably refers to a solvent with a dipole moment (debye, D) greater than 2.0, more preferably greater than 3.0, a relative permittivity of more than 15.0, preferably more than 30.0, and/or to a solvent that is miscible with water. The polar aprotic solvent as disclosed herein is preferably one or more of dimethyl sulfoxide, dichloromethane, tetrahydrofuran, ethyl acetate, acetonitrile, dimethylformamide, dimethyl sulfoxide, acetone, and hexamethylphosphoric triamde, more preferably dimethyl sulfoxide.

Preferably, the “dipole moment” as disclosed herein (is Debye units) is equal to the distance between the charges multiplied by the charge. The dipole moment of a molecule can be calculated by equation (1):

$$\vec{μ} = {\sum }_{i} {q}_{i} {\vec{r}}_{i} i \qquad ( 1 )$$

The “dipole moment” as disclosed herein may be also established by experiment.

The “relative permittivity” as used herein refers to how easily a material can become polarized by imposition of an electric field on an insulator. The relative permittivity is a dimensionless variable and can be determined using equation (2):

$${ε}_{r} = \frac{ε}{{ε}_{0}} \qquad ( 2 )$$

Wherein in equation (2):
εᵣ=relative permittivity
ε=permittivity of substance (C²/(N m²))
ε₀=permittivity of vacuum or free space (8.854187817 10⁻¹² C²/(N m²))

The relative permittivity may change with temperature. Preferably, the relative permittivity is established at 20-25° C. The “relative permittivity” as disclosed herein may be also established by experiment.

The term “miscible with water” as used herein means that the material (typically a liquid) forms a homogeneous mixture when mixed with water. Miscibility of two materials is often determined optically. When the two miscible liquids are combined, the resulting liquid is clear. If the mixture is cloudy the two materials are immiscible. “Miscible” means that no two phases will be produced when mixing the material with water. “Immiscible” means that in some proportions two phases will be produced.

In an embodiment of the method for preparing an antimicrobial formulation as disclosed herein:
the mixture of step c) and/or the antimicrobial formulation has a dynamic viscosity 1 mPa·s-20 Pa·s (pascal-second), preferably 1 mPa·s-2 Pa·s, more preferably 1 mPa·s-200 mPa·s.

The mixture of step c) and/or the antimicrobial formulation in the method as disclosed herein may have a dynamic viscosity of at least 0.01 mPa·s, or at least 0.1 mPa·s, or at least 1 mPa·s, or at least 10 mPa·s, or at least 100 mPa·s, or at least 1 Pa·s, or at least 10 Pa·s, or at least 100 Pas, or at least 1000 Pas. In addition or alternatively, the mixture of step c) and/or the antimicrobial formulation in the method as disclosed herein may have a dynamic viscosity of no more than 1000 Pas, or no more than 100 Pa·s, or no more than 10 Pa·s, or no more than 1 Pa·s, or no more than 100 mPa·s, or no more than 10 mPa·s, or no more than 1 mPa·s, or no more than 0.1 mPa·s, or no more than 0.01 mPa·s.

The term “dynamic viscosity” as used herein relates to a fluid's internal resistance to flow, i.e., relating to the amount of force needed to a fluid flow. The dynamic viscosity is generally expressed in newton-second per square meter (N·s/m2), pascal-second (Pa·s), kilogram per meter per second (kg·m-1·s-1), or poise (P). Preferably, the dynamic viscosity as disclosed herein is measured using a commercially available automated viscometer (e.g., m-VROC, RheoSense, measured using 100 microliter sample volume at a temperature of 20-22° C.).

In an embodiment, the polymer as disclosed herein is a hydrophilic (polar) polymer, such as disclosed herein. In an embodiment, the polymer as disclosed herein is a hydrophobic (non-polar) polymer, such as disclosed herein.

In an embodiment, the aliphatic polyester is a hydrophobic aliphatic polyester, preferably one or more of PDLG (DL-lactide/glycolide copolymer), PLGA (L-lactide/glycolide copolymer), PLC (L-lactide/caprolactone copolymer), PCL polycaprolactone, PD [poly(D-lactide)], PL [poly(L-lactide)], or PDL [(poly(DL-lactide)], more preferably PDLG.

In an embodiment, the polymer is one or more selected from the group consisting of PGA (polyglycolide), poly(ethylene glycol) (PEG), poly(propylene glycol) (PPG), PEG diacrylate (PEGDA), and PEG dimethacrylate (PEGDMA). In an embodiment, the polymer is an aliphatic polyether, for instance, PEG or PPG.

In an embodiment, the aliphatic polyester is not hydrophilic (polar) nor hydrophobic (non-polar).

In an embodiment, the first antibiotic as disclosed herein is one or more of a rifamycin, a hydrophobic hydroquinolone, a macrolide, a tetracycline, or a lincosamide, preferably rifampin.

The rifamycin as taught herein is preferably one or more of rifampin, rifamycin B, rifabutin, rifapentine and rifaximin. The “rifampin” as disclosed herein is also known as rifampicin, therefore “rifampin” may herein be used interchangeable with “rifampicin.” The hydrophobic hydroquinolone as taught herein is preferably one or more of levofloxacin, moxifloxacin. The macrolide as taught herein is preferably one or more of erythromycin, roxithromycin, azithromycin, clarithromycin. The tetracycline as taught herein is preferably one or more of tetracycline, oxytetracycline, doxycycline, tigecycline, minocycline. The lincosamide as taught herein is preferably one or more of lincomycin, clindamycin, pirlimycin.

In an embodiment, the second antibiotic as disclosed herein is one or more of a glycopeptide, an aminoglycoside, a beta-lactam, a carbapenem, a hydrophilic fluoroquinolone, or a streptogramin, preferably vancomycin.

The aminoglycoside as taught herein is preferably one or more of tobramycin, gentamicin, neomycin, streptomycin. The beta-lactam as taught herein is preferably one or more of a penicillin, a cephalosporin, piperacillin. The glycopeptide as taught herein is preferably one or more of vancomycin, teicoplanin, relavancin, ramoplanin. The carbapenem as taught herein is preferably one or more of imipenem, meropenem. The hydrophilic fluoroquinolone as taught herein is preferably one or more of norfloxacin, ciprofloxacin. The streptogramin as taught herein is preferably one or more of pristinamycin, virginiamycin.

In an embodiment, the fluorinated solvent as disclosed herein is a fluorocarbon, a fluoroalcohol, or a mixture thereof, preferably one or more of tetrafluoroethylene, hexafluoroisopropanol, trifluoroethanol, and trifluoropropanol, more preferably tetrafluoroethylene.

In an embodiment of the method for preparing an antimicrobial formulation as disclosed herein:
the second solvent is a non-polar organic solvent, a polar aprotic organic solvent, or a mixture thereof, preferably one or more of chloroform, pentane, hexane, benzene, diethyl ether, and 1,40-Dioxane, more preferably chloroform; and/or
the third solvent is one or more of a polar aprotic organic solvent, preferably one or more of dimethyl sulfoxide, dichloromethane, tetrahydrofuran, ethyl acetate, acetonitrile, dimethylformamide, dimethyl sulfoxide, acetone, and hexamethylphosphoric triamde, more preferably dimethyl sulfoxide.

In an embodiment, the first solvent as disclosed herein is a polar aprotic solvent (such as one or more polar aprotic solvents as disclosed herein).

In an embodiment, the first solvent is a polar protic solvent.

In an embodiment, the first solvent is a non-polar solvent (such as one or more non-polar solvents as disclosed herein).

In an embodiment of the method for preparing an antimicrobial formulation as disclosed herein:
in step a) the polymer is present in the first solvent in a concentration of 50-500 mg/ml, preferably 100-300 mg/ml;
in step b) the first antibiotic is present in the second solvent in a concentration of 25-500 mg/ml, preferably 50-350 mg/ml;
the mixing in step c) is performed at a ratio between second solvent with first antibiotic and first solvent with polymer of 1:1-1:2.5, preferably 1:1.5-1:2;
in step d) the second antibiotic is present in the third solvent in a concentration of 25-500 mg/ml, preferably 50-300 mg/ml; and/or
in step e) the mixing is performed at a ratio between third solvent with second antibiotic and mixture of step c) of 1:2-1:7, preferably 1:3-1:6, more preferably 1:4-1:5.

In step a) of the method for preparing an antimicrobial formulation as disclosed herein, the polymer may be present in the first solvent in a concentration of at least 1 mg/ml, or at least 10 mg/ml, or at least 50 mg/ml, or at least 100 mg/ml, or at least 200 mg/ml, or at least 300 mg/ml, or at least 400 mg/ml, or at least 500 mg/ml, or at least 1 g/ml, or at least 2 g/ml, or at least 5 g/ml, or at least 10 g/ml. In addition or alternatively, the polymer may be present in the first solvent in a concentration of no more than 10 g/ml, or no more than 5 g/ml, or no more than 2 g/ml, or no more than 1 g/ml, or no more than 500 mg/ml, or no more than 400 mg/ml, or no more than 300 mg/ml, or no more than 200 mg/ml, or no more than 100 mg/ml, or no more than 50 mg/ml, or no more than 10 mg/ml, or no more than 1 mg/ml.

In step b) of the method for preparing an antimicrobial formulation as disclosed herein, the first antibiotic may be present in the second solvent in a concentration of at least 1 mg/ml, or at least 10 mg/ml, solvent in a concentration of at least 1 mg/ml, or at least 10 mg/ml, or at least 50 mg/ml, or at least 100 mg/ml, or at least 200 mg/ml, or at least 300 mg/ml, or at least 350 mg/ml, or at least 400 mg/ml, or at least 500 mg/ml, or at least 1 g/ml, or at least 2 g/ml, or at least 5 g/ml, or at least 10 g/ml. In addition or alternatively, the first antibiotic may be present in the second solvent in a concentration of no more than 10 g/ml, or no more than 5 g/ml, or no more than 2 g/ml, or no more than 1 g/ml, or no more than 500 mg/ml, or no more than 400 mg/ml, or no more than 350 mg/ml, or no more than 300 mg/ml, or no more than 200 mg/ml, or no more than 100 mg/ml, or no more than 50 mg/ml, or no more than 10 mg/ml, or no more than 1 mg/ml.

In step c) of the method for preparing an antimicrobial formulation as disclosed herein, the mixing in step c) may be performed at a ratio between second solvent with first antibiotic and first solvent with polymer of at least 1:1, or at least 1:1.5, or at least 1:2, or at least 1:2.5, or at least 1:3, or at least 1:3.5, or at least 1:4, or at least 1:4.5, or at least 1:5, or at least 1:6, or at least 1:7, or at least 1:8, or at least 1:9, or at least 1:10, or at least 1:20. In addition or alternatively, the mixing in step c) may be performed at a ratio between second solvent with first antibiotic and first solvent with polymer of no more than 1:20, or no more than 1:10, or no more than 1:9, or no more than 1:8, or no more than 1:7, or no more than 1:6, or no more than 1:5, or no more than 1:4.5, or no more than 1:4, or no more than 1:3.5, or no more than 1:3, or no more than 1:2.5, or no more than 1:2, or no more than 1:1.5, or no more than 1:1.

In step d) of the method for preparing an antimicrobial formulation as disclosed herein, the second antibiotic may be present in the third solvent in a concentration of at least 1 mg/ml, or at least 10 mg/ml, or at least 50 mg/ml, or at least 100 mg/ml, or at least 200 mg/ml, or at least 300 mg/ml, or at least 400 mg/ml, or at least 500 mg/ml, or at least 1 g/ml, or at least 2 g/ml, or at least 5 g/ml, or at least 10 g/ml. In addition or alternatively, second antibiotic may be present in the third solvent in a concentration of no more than 10 g/ml, or no more than 5 g/ml, or no more than 2 g/ml, or no more than 1 g/ml, or no more than 500 mg/ml, or no more than 400 mg/ml, or no more than 300 mg/ml, or no more than 200 mg/ml, or no more than 100 mg/ml, or no more than 50 mg/ml, or no more than 10 mg/ml, or no more than 1 mg/ml.

In step e) of the method for preparing an antimicrobial formulation as disclosed herein, the mixing is performed at a ratio between third solvent with second antibiotic and mixture of step c) of at least 1 mg/ml, or at least 10 mg/ml, or at least 50 mg/ml, or at least 100 mg/ml, or at least 200 mg/ml, or at least 300 mg/ml, or at least 400 mg/ml, or at least 500 mg/ml, or at least 1 g/ml, or at least 2 g/ml, or at least 5 g/ml, or at least 10 g/ml. the mixing is performed at a ratio between third solvent with second antibiotic and mixture of step c) of no more than 10 g/ml, or no more than 5 g/ml, or no more than 2 g/ml, or no more than 1 g/ml, or no more than 500 mg/ml, or no more than 400 mg/ml, or no more than 300 mg/ml, or no more than 200 mg/ml, or no more than 100 mg/ml, or no more than 50 mg/ml, or no more than 10 mg/ml, or no more than 1 mg/ml.

In an aspect, the current disclosure relates to an antimicrobial formulation, comprising:
a polymer at a concentration of 10-300 mg/ml of the antimicrobial formulation, preferably 50-150 mg/ml, wherein the polymer is an aliphatic polyester, preferably a hydrophobic aliphatic polyester, more preferably one or more of PDLG (DL-lactide/glycolide copolymer), PLGA (L-lactide/glycolide copolymer), PLC (L-lactide/caprolactone copolymer), PCL polycaprolactone, PD [poly(D-lactide)], PL [poly(L-lactide)], or PDL [(poly(DL-lactide)], most preferably PDLG;
a first antibiotic at a concentration of 10-200 mg/ml of the antimicrobial formulation, preferably 20-100 mg/ml, wherein the first antibiotic is hydrophobic and/or soluble in a non-polar organic solvent, preferably one or more of a rifamycin, a hydrophobic hydroquinolone, a macrolide, a tetracycline, or a lincosamide, more preferably rifampin;
a second antibiotic at a concentration of 10-200 mg/ml of the antimicrobial formulation, preferably 20-100 mg/ml, wherein the second antibiotic is hydrophilic and/or soluble in a polar aprotic organic solvent, preferably one or more of a glycopeptide, an aminoglycoside, a beta-lactam, a carbapenem, a hydrophilic fluoroquinolone, or a streptogramin, more preferably vancomycin;
a non-polar organic solvent at a concentration of 10-50% (v/v) with respect to the antimicrobial formulation, preferably 20-40% (v/v), wherein the non-polar organic solvent is preferably one or more of chloroform, pentane, hexane, benzene, diethyl ether, and 1,40-Dioxane, more preferably chloroform; and/or
a polar aprotic organic solvent at a concentration of 1-40% (v/v) with respect to the antimicrobial formulation, preferably 10-30% (v/v), wherein the polar aprotic solvent is preferably one or more of dimethyl sulfoxide, dichloromethane, tetrahydrofuran, ethyl acetate, acetonitrile, dimethylformamide, dimethyl sulfoxide, acetone, and hexamethylphosphoric triamde, more preferably dimethyl sulfoxide.

In the antimicrobial formulation of the current disclosure, the polymer, first antibiotic, and/or second antibiotic may be present in an amount of at least 1 mg/ml, or at least 10 mg/ml, solvent in a concentration of at least 1 mg/ml, or at least 10 mg/ml, or at least 50 mg/ml, or at least 100 mg/ml, or at least 200 mg/ml, or at least 300 mg/ml, or at least 350 mg/ml, or at least 400 mg/ml, or at least 500 mg/ml, or at least 1 g/ml, or at least 2 g/ml, or at least 5 g/ml, or at least 10 g/ml. In addition or alternatively, the polymer, first antibiotic, and/or second antibiotic may be present in an amount of no more than 10 g/ml, or no more than 5 g/ml, or no more than 2 g/ml, or no more than 1 g/ml, or no more than 500 mg/ml, or no more than 400 mg/ml, or no more than 350 mg/ml, or no more than 300 mg/ml, or no more than 200 mg/ml, or no more than 100 mg/ml, or no more than 50 mg/ml, or no more than 10 mg/ml, or no more than 1 mg/ml.

In the antimicrobial formulation of the current disclosure, the non-polar organic solvent may be present in a concentration (v/v with respect to the antimicrobial formulation) of at least 1%, or at least 2%, or at least 5%, or at least 10%, or at least 20%, or at least 30%, or at least 40%, or at least 50%, or at least 60%, or at least 70%, or at least 80%, or at least 90%. In addition or alternatively, the non-polar organic solvent may be present in a concentration (v/v with respect to the antimicrobial formulation) of no more than 90%, or no more than 80%, or no more than 70%, or no more than 60%, or no more than 50%, or no more than 40%, or no more than 30%, or no more than 20%, or no more than 10%, or no more than 5%, or no more than 2%, or no more than 1%.

In an embodiment, the antimicrobial formulation as disclosed herein has a dynamic viscosity 1 mPa·s-20 Pas (pascal-second), preferably 1 mPa·s-2 Pa·s, more preferably 1 mPa·s-200 mPa·s.

The antimicrobial formulation as disclosed herein may have a dynamic viscosity of at least 0.01 mPa·s, or at least 0.1 mPa·s, or at least 1 mPa·s, or at least 10 mPa·s, or at least 100 mPa·s, or at least 1 Pa·s, or at least 10 Pas, or at least 100 Pa·s, or at least 1000 Pa·s. In addition or alternatively, the antimicrobial formulation as disclosed herein may have a dynamic viscosity of no more than 1000 Pas, or no more than 100 Pa·s, or no more than 10 Pas, or no more than 1 Pa·s, or no more than 100 mPa·s, or no more than 10 mPa·s, or no more than 1 mPa·s, or no more than 0.1 mPa·s, or no more than 0.01 mPa·s.

In an aspect, the current disclosure relates to the use of the antimicrobial formulation (obtainable by the method disclosed herein) for preparing an antimicrobial coating. Preparing an antimicrobial coating herein is preferably by dip coating, spin coating, spraying, electrospinning, electrophoretic deposition, sputter coating, thermal spraying, plasma spraying, sol-gel, or layer-by-layer coating, more preferably electrospraying.

In an aspect, the current disclosure relates to a method for preparing an antimicrobial coating, the method comprising applying the antimicrobial formulation obtainable by one of the various embodiments of the method for preparing an antimicrobial formulation as disclosed herein on a substrate and removing the one or more solvents in the antimicrobial formulation. In the method for preparing an antimicrobial coating, preferably the applying is performed by dip coating, spin coating, spraying, electrospinning, electrophoretic deposition, sputter coating, thermal spraying, plasma spraying, sol-gel, layer-by-layer coating, more preferably electrospraying.

The term “electrospraying” as disclosed herein relates to a method wherein a liquid (e.g., an antimicrobial formulation) is deposited onto a substrate using an electrostatic force and the liquid's surface tension to produce one or more generations of charged, monodisperse droplets that are directed toward the substrate. Typically, the electrospray process uses a high-voltage power supply, container (e.g., syringe) capped by a metallic capillary to hold a liquid, pump to control the flow of the liquid, and a grounded collector (e.g., metallic substrate). When a high electric field is applied at the needle, a charged liquid jet will break up into droplets. At the tip of the nozzle, the charged solution may break into micro-scale droplets. Furthermore, child droplets may form that possess larger surface to volume ratios. This process may occur multiple times depending on the spray distance and solution composition, resulting in several generations of monodisperse droplets, most typically two. This eventually forms small particles with generally narrow size distribution on the collector. “Electrospraying” as disclosed herein may be used interchangeable with “electrospray deposition” or “electrodynamic spraying.” The skilled person is well aware of the variations in electrospraying techniques and the applications thereof (e.g., Wang et al. Electrospraying: Possibilities and Challenges of Engineering Carriers for Medical Applications-A Mini Review. Front. Chem., 25 Apr. 2019).

Preferably, the electrospraying as disclosed herein uses the following, or similar, protocol: the antimicrobial formulation is deposited into a syringe (e.g., a 3 mL-5 mL) with a needle (e.g., steel). A voltage is applied between the needle of the and the (metal object to be coated to create the required electric field. The voltage is preferably 10-30 kV (e.g., 16 kV). The coating solution is fed through the syringe needle (e.g., using a syringe pump), preferably with a flow rate of 0.1-1 ml/h (e.g., 0.5 ml/h). Electrosprayed drops are deposited onto the (metal) object to be coated placed at an appropriate distance from the needle tip, preferably 5-15 cm (e.g., 8 cm). The coated objects may be additionally treated to remove organic solvents (e.g., by keeping in vacuum with cold trap, liquid nitrogen).

The “substrate” as disclosed herein may be a metal, polymer, glass, bioglass, and/or elastomer, preferably a metal, more preferably stainless steel (e.g., surgical grade stainless steel such as 316L), cobalt-chromium (Co—Cr) alloys, pure commercial titanium (Ti), or a Ti alloy. The substrate is preferably a surface of a medical device. The “medical device” as disclosed herein is typically a device capable of being or designed to be implanted in a subject's body as part of medical diagnosis and/or treatment. The medical device may be degradable or non-degradable. Common examples of medical devices are: stents, catheters, pacemakers, implantable cardiac defibrillators, coronary stents, degradable grafts, ear tubes, interocular lenses, implantable insulin pumps, intra-uterine devices, surgical mesh implants, breast implants, prosthesis, orthopedic implants, artificial joints. The term “medical device” may herein be used interchangeably with “biomedical device,” “implantable device,” “implant,” or “(bio) medical implant.”

The “removing” of a solvent in a material as disclosed herein—e.g., removing in an antimicrobial formulation or removing in an antimicrobial coating—may involve any method that decreases the amount of solvent in the material to a specified level. The removal of the solvent is preferably done by evaporating the solvent. A preferred method of evaporation involves leaving the material at room temperature (20-22° C.) until the amount of residual solvent in the material has decreased to a specified level. Another preferred method is to dry the material at elevated temperatures (e.g., 22-50° C.) until the amount of solvent in the material has decreased to a specified level. In addition or alternative, the material may be placed in a compressed gas (e.g., argon, nitrogen, helium or air), for example, to help evaporating the solvent, until the amount of solvent in the material has decreased to a specified level. The specified level of residual solvent is typically the level at which the solvent is considered safe and induce no harmful effects in the human body. The skilled person is well aware that the amount of residual solvent acceptable in a material for use in the human body is dependent on the type of solvent and the therapeutic use of the material carrying the residual solvent.

In an aspect, the current disclosure relates to an antimicrobial coating, comprising:
a polymer in an amount of 40-90%, preferably 50-80%, by weight of the antimicrobial coating, wherein the polymer is an aliphatic polyester, preferably a hydrophobic aliphatic polyester;
a first antibiotic in an amount of 5-40%, preferably 10-30%, by weight of the antimicrobial coating, wherein the first antibiotic is hydrophobic and/or soluble in a non-polar organic solvent; and
a second antibiotic in an amount of 5-40%, preferably 10-30%, by weight of the antimicrobial coating, wherein the second antibiotic is hydrophilic and/or soluble in a polar aprotic organic solvent,
wherein the first antibiotic and the second antibiotic are comprised in a matrix effected by the polymer.

In the antimicrobial coating as disclosed herein, the polymer, first antibiotic, and/or second antibiotic may be (individually) present in an amount (by weight of the antimicrobial coating) of at least 1%, or at least 5%, or at least 10%, or at least 20%, or at least 30%, or at least 40%, or at least 50%, or at least 60%, or at least 70%, or at least 80%, or at least 90%. In addition or alternatively, the polymer, first antibiotic, and/or second antibiotic may be (individually) present in an amount (by weight of the antimicrobial coating) of no more than 90%, or no more than 80%, or no more than 70%, or no more than 60%, or no more than 50%, or no more than 40%, or no more than 30%, or no more than 20%, or no more than 10%, or no more than 5%, or no more than 1%.

In an embodiment of the antimicrobial coating as disclosed herein:
the aliphatic polyester is one or more of PDLG (DL-lactide/glycolide copolymer), PLGA (L-lactide/glycolide copolymer), PLC (L-lactide/caprolactone copolymer), PCL polycaprolactone, PD [poly(D-lactide)], PL [poly(L-lactide)], or PDL [(poly(DL-lactide)], preferably PDLG;
the first antibiotic is one or more of a rifamycin, a hydrophobic hydroquinolones, a macrolide,
a tetracycline, or a lincosamide, preferably rifampin; and/or
the second antibiotic is one or more of a glycopeptide, an aminoglycoside, a beta-lactam, a carbapenem, a hydrophilic fluoroquinolone, or a streptogramin, preferably vancomycin.

In an embodiment of the antimicrobial coating as disclosed herein:
the antimicrobial coating is a single layer antimicrobial coating, preferably with a thickness of 10-1000 μm, preferably 50-500 μm, more preferably 100-250 um;
the total polymer content in the antimicrobial coating is effected for 90-100 wt. % by a single polymer, preferably 95-100 wt. %, more preferably 99-100 wt. %, most preferably 100 wt. %; and/or
the surface of the antimicrobial coating is hydrophobic, as defined by a static water contact angle of 70 or more, preferably 80 or more, more preferably 90 or more; and/or
the antimicrobial coating has an average surface roughness of 20-1000 nm, preferably 20-500 nm (e.g., 50-400 nm, or 100-300 nm), more preferably 20-200 nm.

The antimicrobial coating as disclosed herein may have a thickness of at least 1 μm, or at least 5 μm, or at least 10 μm, or at least 20 μm, or at least 30 μm, or at least 40 μm, or at least 50 μm, or at least 60 μm, or at least 70 μm, or at least 80 μm, or at least 90 μm, or at least 100 μm, or at least 200 μm, or at least 300 μm, or at least 400 μm, or at least 500 μm, or at least 600 μm, or at least 700 μm, or at least 800 μm, or at least 900 μm, or at least 1000 μm, or at least 2000 μm, or at least 3000 μm, or at least 4000 μm, or at least 5000 μm. In addition or alternatively, the antimicrobial coating as disclosed herein may have a thickness of no more than 5000 μm, or no more than 4000 μm, or no more than 3000 μm, or no more than 2000 μm, or no more than 1000 μm, or no more than 900 μm, or no more than 800 μm, or no more than 700 μm, or no more than 600 μm, or no more than 500 μm, or no more than 400 μm, or no more than 300 μm, or no more than 200 μm, or no more than 100 μm, or no more than 90 μm, or no more than 80 μm, or no more than 70 μm, or no more than 60 μm, or no more than 50 μm, or no more than 40 μm, or no more than 30 μm, or no more than 20 μm, or no more than 10 μm, or no more than 5 μm, or no more than 1 μm.

The “thickness” of the antimicrobial coating as used herein relates to the average thickness, such as determined for at least 1 mm², preferably at least 1 cm² of the antimicrobial coating. The average thickness may be based on sufficient measurements performed on a (part of the) sample to reduce the standard deviation to no more than 20%. The thickness of the antimicrobial coating as disclosed herein is preferably measured by spectroscopic reflectometry. Spectroscopic reflectometry typically measures the amount of light reflected from a thin film over a range of wavelengths, with the incident light normal to the sample surface. In a preferred protocol, the thickness of the coatings is measured using a Filmetrics thin film measurement system (e.g., F40, Filmetrics Europe GmbH, or similar) set up on an incident light microscope using the wavelength range from 400 to 850 nm. The optical index of refraction is preferably assumed to be n=1.5.

The term “roughness” as used herein relates to the surface roughness of the coating. The “roughness” preferably relates to the mean roughness (e.g., based on sufficient measurements performed on a sample to reduce the standard deviation to no more than 20%). The roughness of the antimicrobial coating as disclosed herein is preferably measured by atomic force microscopy (AFM). In a preferred protocol, the roughness of a surface is measured using an atomic force microscopy (AFM, Easy Scan, or similar) recording in tapping mode at room temperature in air.

It was found that the antimicrobial formulation of the disclosure allows the formation of a coating with no or reduced porosity, meaning reduced pore size and/or reduced pore diameter. It was possible to achieve a coating with no apparent porosity.

In an embodiment, the antimicrobial coating has a density or a morphology, which can be defined by the porosity disclosed herein.

The low/absence of porosity of the coating may offer one or more benefits in terms of, among others:
release of antibiotics, for instance, more sustained release of antibiotics;
more uniform coating;
fewer air pockets;
more firm attachment to the implant material, e.g., ameliorating delamination during implantation;
thinner, e.g., allowing easier implantation;
improved attachment of cells;
higher drug loading (concentration);
higher polymer loading (concentration).

In a preferred embodiment, the antimicrobial coating as disclosed herein has:
a porosity of no more than 20%, or no more than 10%, or no more than 1%, or no more than 0.1%; and/or
a pore diameter of no more than 1 μm, preferably of no more than 0.1 μm, more preferably of no more than 0.01 μm.
wherein the antimicrobial formulation is obtainable by the method as disclosed herein.

In a preferred embodiment, the antimicrobial coating as disclosed herein has no porosity.

The term ‘porosity’ of the coating as used herein means the fraction of the volume devoid of (solid) coating (e.g., the voids or empty spaces formed by air or other gas) over the total volume of the coating. The porosity is typically expressed as the volume fraction, ranging between 0 and 1, or as a percentage between 0 and 100%. In the context of the current disclosure, the porosity is preferably expressed as a percentage (%). The person skilled in the art is familiar with several methods to measure the porosity of a material. Preferred methods for measuring the porosity (or pore diameter) include mercury porosimetry, liquid intrusion, gravimetry and microscopy techniques (e.g., Confocal Laser Scanning Microscopy, Confocal Laser Scanning Microscopy, Scanning electron microscopy). Preferred protocols for determining porosity (or pore diameter) by liquid intrusion, gravimetry and scanning electron microscopy (SEM) are described by Soliman et al. (Acta Biomaterialia 6 (2010) 1227-1237). In a preferred liquid intrusion protocol, the coated samples are weighed prior to immersion in ethanol (intruding liquid of density ρ_{EtOH}=0.789 g/ml), left overnight on a shaker table to allow diffusion of ethanol into the void volume, blotted with a Kimwipe and reweighed. The porosity is calculated as ε=V_{EtOH}/(V_{EtOH}+V_{coating}) by dividing the volume V_{EtOH} of the intruded ethanol by the total volume after intrusion. In a preferred gravimetry protocol, the porosity is evaluated as ε=1 ρ_{APP}/ρ_{PCL}, where the apparent coating density ρ_{APP} is measured as the mass to volume ratio. In a preferred SEM protocol, the porosity ε=VF is determined as the average projected porosity deduced from the percent void fraction VF as seen in top view SEM micrographs of the samples. The percent VF is determined by counting and summing the through thickness voids in a 2D SEM image. For instance, a Matlab (MathWorks Inc.) script can be used to convert grey scale images into a black a white format and to compute the void fraction (i.e., the fraction of white pixels) in an automated fashion. The term “porosity” as used herein preferably means the average porosity, such as determined for a coating surface area of 0.5 cm² and/or a coating thickness of 100 μm, or as determined for a dry weight amount of coating of ˜50 mg.
The term ‘pore diameter’ as used herein means the diameter of the pores in a porous structure, for instance, a polymeric coating. There is no unique definition of pore diameter since it depends on the measurement technique and whether it is assessed following a 2-dimensional or a 3-dimensional approach (Bartoš et al. Biomed Eng Online. 2018 Aug. 17; 17(1):110). The pore diameter as taught herein is determined by means of scanning electron microscopy (SEM), e.g., as follows: using image analysis software (for example, using the manual mode of freeware ImageJ software, US National Institutes of Health). 40 pores are assessed for each of the two perpendicular sections, and repeated for a total of 4 SEM micrographs (imaged at 100×) for each perpendicular section. Randomly selected pores are analyzed for the long diameter (longest wall-to-wall distance across the pore) and the short diameter (shortest wall-to-wall distance across the pore). The average of the long and short diameters determines the pore diameter. The “pore diameter” as used herein preferably means the average pore diameter, such as determined for a coating surface area of 0.5 cm² and/or a coating thickness of 100 μm, or as determined for a dry weight amount of coating of ˜50 mg. The term “pore diameter” in the context of the current disclosure can be used interchangeably with the term “pore size.”

In an embodiment, the antimicrobial coating as disclosed herein has a porosity of not more than 75%, not more than 70%, not more than 65%, not more than 60%, not more than 55%, not more than 50%, not more than 45%, not more than 40%, not more than 35%, not more than 30%, not more than 25%, not more than 20%, not more than 15%, not more than 10%, not more than 5%, not more than 1%, not more than 0.5%, not more than 0.1%, not more than 0.05%, not more than 0.01%, or not more than 0.001%. Preferably, the antimicrobial coating as disclosed herein has a porosity of 0.0001%-10%, more preferably 0.001-1%, even more preferably 0.01-0.1%. In an embodiment, the antimicrobial coating as disclosed herein has no porosity (i.e., 0%).

In an embodiment, the antimicrobial coating as disclosed herein has an average pore diameter of not more than 100 μm, not more than 50 μm, not more than 25 μm, not more than 10 μm, not more than 5 μm, not more than 1 μm, not more than 0.5 μm (500 nm), not more than 0.4 μm (400 nm), not more than 0.3 μm (300 nm), not more than 0.2 μm (200 nm), not more than 0.1 μm (100 nm), not more than 0.05 μm (50 nm), not more than 0.01 μm (10 nm), or nor more than 0.001 (1 nm). Preferably, the antimicrobial coating as disclosed herein has a pore diameter of 0.00001-1 μm, more preferably 0.0001-0.1 μm, more preferably 0.001-0.01 μm 0.1%. In an embodiment, the antimicrobial coating as disclosed herein has no pore diameter (0 μm).

In an aspect, the current disclosure relates to an antimicrobial formulation as disclosed herein, an antimicrobial formulation obtainable by a method as disclosed herein, an antimicrobial coating as disclosed herein, and/or an antimicrobial coating obtainable by a method as disclosed herein, for use in the treatment and/or prevention of medical device-related infections, preferably the treatment and/or prevention of orthopedic implant-related infections.

The antimicrobial formulation is preferably obtainable or obtained by the method as disclosed herein. The antimicrobial coating is preferably obtainable or obtained by coating with the antimicrobial formulation as disclosed herein.

The “medical device-related infection” and/or “orthopedic implant-related infection” as disclosed herein is preferably an infection caused by a micro-organisms, typically yeasts (E.g., Candida albicans) or bacteria such as Staphylococcus ssp. (e.g., Staphylococcus aureus, Staphylococcus epidermidis) Streptococcus ssp. (e.g., Streptococcus bovis), Enterococcus species, and (other) Gram-negative bacilli such as Klebsiella, Enterobacter, Acinetobacter, Pseudomonas, and Escherichia ssp. The medical device-related infection and/or orthopedic implant-related infection as disclosed herein may be an infection caused by an antibiotic-resistant strain, such as vancomycin-resistant Enterococcus, methicillin-resistant Staphylococcus aureus, methicillin-resistant Staphylococcus epidermidis, and multidrug-resistant gram-negative bacilli. The infection in a “medical device-related infection” and/or “orthopedic implant-related infection may be present on the surface of the medical device/implant, typically in the form of a biofilm. In addition or alternatively, the infection may be present in the tissue around the medical device/implant. The medical device-related infection” and/or “orthopedic implant-related infection” may be the result of the surgical procedure (e.g., the presence of an open wound through which micro-organisms are introduced into the body) and/or the result of the presence of the medical device/implant in the body (e.g., micro-organisms may adhere to the medical device/implant or micro-organisms may more readily survive in the tissue around the medical device/implant such as due to tissue damage and/or a change in the immune response).

The “orthopedic implant” as disclosed herein can be one or more of a prosthesis, bone screw, bone pin, hook, rod, bone substitute, internal fixator, external fixator, intramedullary nail, K-wire, spacer, cage, prosthetic frame, anchor, arthrodesis nail, and bone plate. The orthopedic implant of the present technology can comprise a solid metal, for example, gold, silver, stainless steel, platinum, palladium, iridium, iron, nickel, copper, titanium, aluminum, chromium, cobalt, molybdenum, vanadium, tantalum, and alloys thereof. In preferred embodiments, the orthopedic implant comprises a metal including surgical stainless steel, titanium and/or a titanium alloy. The orthopedic implant may be a solid implant or a (partially) porous implant, such as produced by selective laser melting, metal 3D printing, direct metal laser sintering, and/or additive manufacturing.

The “orthopedic-implant related infection” as used herein may be an infection that has newly established in a subject, i.e., the subject did not have or was not previously diagnosed with the infection. The “orthopedic-implant related infection” may also be an infection that was not successfully treated by systemic antibiotics alone or other therapy, so that a revision surgery is required. For example, the prevention and/or treatment of the orthopedic-implant related infection may be part of a revision surgery wherein the original implant is removed and a new implant (preferably comprising the antimicrobial coating as disclosed herein) is introduced. The revision surgery may be a one-stage or a two-stage procedure. In a one-stage revision surgery, typically the infected orthopedic implant is removed, the infected site is irrigated, and a new orthopedic implant is inserted. In a two-stage revision surgery, typically a first surgery is performed for removing the infected orthopedic implant, and implanting an antibiotic loaded spacer. The spacer is left in the subject for typically for several weeks (e.g., 4-8 weeks, usually 6 weeks) while the subject may receive systemic antibiotic administration. After the infection is cleared, a second surgery is performed in which the spacer is removed and the new orthopedic implant is introduced. The antimicrobial coating as disclosed herein may be present on the orthopedic implant as part of a primary surgery (patient has no infection yet), as part of a one-stage revision surgery, and/or as part of a two-stage revision surgery. In an embodiment, the antimicrobial coating is present on a spacer, such as used in a two-stage revision surgery. In accordance with the foregoing, the term “implant” may be used interchangeably herein with “spacer.”

In an embodiment of the antimicrobial coating disclosed herein:
the amount of first and second antibiotic together is 20%-60%, preferably 30%-50%, by weight of the antimicrobial coating; and/or
the ratio between first and second antibiotic together to polymer is 1:1-1:3, preferably 1:1.5-1:2.5.
In the antimicrobial coating as disclosed herein, the ratio between first and second antibiotic together to polymer is at least 1:1, or at least 1:1.5, or at least 1:2, or at least 1:2.5, or at least 1:3, or at least 1:3.5, or at least 1:4, or at least 1:4.5, or at least 1:5, or at least 1:6, or at least 1:7, or at least 1:8, or at least 1:9, or at least 1:10, or at least 1:20. In addition or alternatively, the ratio between first and second antibiotic together to polymer is no more than 1:20, or no more than 1:10, or no more than 1:9, or no more than 1:8, or no more than 1:7, or no more than 1:6, or no more than 1:5, or no more than 1:4.5, or no more than 1:4, or no more than 1:3.5, or no more than 1:3, or no more than 1:2.5, or no more than 1:2, or no more than 1:1.5, or no more than 1:1.

In the antimicrobial coating as disclosed herein, the amount of first and second antibiotic together may be (by weight of the antimicrobial coating).

In an aspect, the current disclosure relates to the use of an antibiotic and/or a solvent as a viscosity-reducing agent in the preparation of an antimicrobial formulation, wherein:
the antibiotic is preferably hydrophobic and/or preferably is soluble in a non-polar organic solvent, preferably one or more of a rifamycin, a hydrophobic hydroquinolones, a macrolide, a tratracycline, or a lincosamide, more preferably rifampin;
the solvent is preferably a non-polar organic solvent, more preferably one or more of chloroform, pentane, hexane, benzene, diethyl ether, and 1,40-Dioxane, most preferably chloroform; and/or
the antimicrobial formulation preferably is for use as a coating formulation, more preferably in dip coating, spin coating, spraying, electrospinning, electrophoretic deposition, sputter coating, thermal spraying, plasma spraying, sol-gel, layer-by-layer coating, most preferably electrospraying.

In addition or alternatively, the use of an antibiotic and/or a solvent as disclosed herein, may comprise:
a first antibiotic, which is preferably hydrophobic and/or preferably is soluble in a non-polar organic solvent, preferably one or more of a rifamycin, a hydrophobic hydroquinolones, a macrolide, a tratracycline, or a lincosamide, more preferably rifampin; and
a second antibiotic, which is preferably hydrophilic and/or preferably soluble in a polar aprotic organic, preferably one or more of a glycopeptide, an aminoglycoside, a beta-lactam, a carbapenem, a hydrophilic fluoroquinolone, or a streptogramin, more preferably vancomycin.

The terms ‘comprising’ or ‘to comprise’ and their conjugations, as used herein, refer to a situation wherein the terms are used in their non-limiting sense to mean that items following the word are included, but items not specifically mentioned are not excluded. It also encompasses the more limiting verb ‘to consist essentially of’ and ‘to consist of “.

Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one’.

The terms ‘to increase’ and ‘increased level’ and the terms ‘to decrease’ and ‘decreased level’ refer to the ability to significantly increase or significantly decrease or to a significantly increased level or significantly decreased level. Generally, a level is increased or decreased when it is at least 5%, such as 10%, 15%, 20%, 25%, 30%, 35%, 40%, 45%, 50% higher or lower, respectively, than the corresponding level in a control or reference. Alternatively, a level in a sample may be increased or decreased when it is statistically significantly increased or decreased compared to a level in a control or reference.

### EXPERIMENTAL EXAMPLES

### Example 1

### 1. Aim

The aim of this study was to determine the parameters required for preparing an antimicrobial coating, wherein the coating preferably 1) is easily adaptable to incorporate different antibiotic of choice (preferably hydrophobic/non-polar and hydrophilic/polar drug combinations), 2) can be applied as a single polymer thin film coating, 3) can incorporate high drug content(s), and 4) can have sufficient low viscosity to be applied by different coating techniques, including electrospray.

The general protocol for manufacturing the antimicrobial coating was as follows:
1. dissolving the resorbable polymer and antibiotic(s) each separate in an appropriate solvent;
2. mixing the polymer solution with the antibiotic solution(s) to obtain a polymer-antibiotic solution;
3. applying the polymer-antibiotic solution as coating on a medical device;
4. evaporating the solvents.

The following main parameters, and their combinations, were evaluated for their effect on coatability of the coating solutions, drug release profile, and the antimicrobial effect:
type of solvent (e.g., non-polar vs. polar organic solvent);
type of antibiotic (e.g., hydrophobic/non-polar vs. hydrophilic/polar antibiotic);
sequence of mixing the polymeric, first antibiotic, and second antibiotic solutions;
type of resorbable polymer (e.g., polyester vs. non-polyester); and
the optimal drug/polymer content and ratio.

Drug solubility and viscosity were used as main criteria to determine the coatability of the various coating solutions. Electrospraying was chosen as the preferred coating method, since it a technically challenging method generally requiring low viscosity coating solutions, relative to other coating methods.

### 2. Methods

### 2.1. Chemicals

| | Hydrophobic | Hydrophilic |
|---|---|---|
| Polyester (co)polymers | antibiotics | antibiotics |
| PDLG | Rifampicin | Vancomycin |
| DL-lactide/glycolide copolymer | Erythromycin | Gentamycin |
| (PDLG 50:50 monomer ratio) | Minocycline | Tobramycin |
| PLC | | |
| L-lactide/caprolactone | | |
| copolymer | | |
| PLDL | | |
| L-lactide/DL-lactide copolymer | | |
| PCL | | |
| Polycaprolactone | | |
| PL | | |
| Poly(L-lactide) | | |
| PEG | | |
| Polyethylene glycol | | |

### 2.2. Preparation of the Antimicrobial Formulation

All the following steps were done at room temperature under the chemical fume hood.
1. A polymeric solution (solution A) was prepared by dissolving an aliphatic polyester in tetrafluoroethylene (TFE, Sigma, Germany) under stirring for 12 h to reach a concentration of 200 mg/ml.
2. A first antibiotic solution (solution B) was prepared dissolving a first antibiotic (hydrophilic or hydrophobic) in chloroform (VWR, France) or dimethyl sulfoxide (DMSO, Merck, Germany) under stirring to reach a concentration of 50-250 mg/ml.
3. A viscosity-reducing drug-polymer solution (solution C) was prepared by adding solution B dropwise to A with a ratio of 30-40% (v/v) and stirring the mixture under stirring for 6 hours.
4. A second antibiotic solution (solution D) was prepared by dissolving a second antibiotic (hydrophilic) in dimethyl sulfoxide at 40° C. under stirring to reach a concentration of 50-250 mg/ml.
5. The coating solution (solution E) was prepared by adding solution D dropwise to solution C with a ratio of 15-20% (v/v) under stirring for 24 hours.

### 2.3. Coating

Titanium discs (diameters of 8 mm, height of 4 mm) were produced from commercial pure titanium (CP-Ti) powder (grade 1, medical grade quality, LPW, UK). The titanium discs were coated by electrospraying, electrospinning or spin coating.

Electrospraying and electrospinning comprised a home-made uniaxial solution electrospinning system. Briefly, the drug-polymer solution was loaded into 3 ml syringes fitted with a 27-gauge (G) needle. A voltage of 16-20 kV (Heinzinger, Germany) was applied between the needle and the implant. A syringe pump (World precision instruments, US) was used to feed the solution through the needle at a flow rate of 0.5-1 ml/h. The polymeric solution was sprayed directly onto the implants placed at a distance of 8 cm from the needle. The coated implants were kept in vacuum with cold trap (liquid nitrogen) for 3 days to remove all organic solvents.

The spin coating process was performed at room temperature at different spin speed rates, in the range of 200-600 rpm using a spin coater (450 SPIN COATER, Netherlands) In all of the coating experiments, a volume of 20 μL of final solution was dropped in the middle of the implant before the rotation process had started. Then, the spinner was turned on, producing a layer of coating on the surface of implant. This process was continued until formation of desired coating. After coating, the samples were dried under vacuum for 3 days.

It was found that the different coating methods deposited a total 5-15 mg coating, as determined by weighing the discs before and after coating.

### 2.4. Viscosity Measurement

The dynamic viscosity of the antimicrobial formulations was measured using the m-VROC viscometer (RheoSense).

### 2.5. Drug Release Profile

The antibiotics release kinetics were characterized by incubating the implants in PBS (pH 7.4) at 37° C. The supernatant was refreshed after on the days as indicated in the figures, and stored at −20° C. until analysis. Concentrations of drugs in the release media were measured using an ACQUITY ssUPLC H-Class PLUS Bio system (Waters Corporation, US) equipped with a BEH C18 Column (1.7 μm, 2.1 mm×50 mm) and a Waters 2996 PDA detector. Concentrations of the drugs were quantified using wavelengths for Vancomycin (233 nm) and Rifampin (264 nm) and normalized against a calibration curve obtained by diluting stock solutions of Rif (1 mg/ml in ACN) and Van (1 mg/ml in MQ) in PBS.

### 2.6. Zone of Inhibition

The implants were incubated for four or six weeks in PBS at 37° C. to allow elution of the antibiotics. The presence and amount of drug elution at week four and week six was measured in terms of the zone of inhibition on bacterial growth. To this end, implants were placed on trypticase soy agar plates that were inoculated with 0.5 ml bacterial suspension prepared at OD₆₀₀=0.01 (about 10⁷ CFU/ml) to provide a bacterial lawn. After 24 h at 37° C., the zone of inhibition of bacterial growth was measured (expressed in cm).

### 3. Results

### 3.1. Coatability of Different Antimicrobial Formulations

The suitability of several (multidrug) coating solutions as to form a thin film coating by means of electrospraying was determined. It was found that the presence of a hydrophobic antibiotic (rifampicin, erythromycin, minocycline) and/or a non-polar solvent (chloroform) was important to obtain a homogenous and low viscosity solution (Table 1). Coating solutions lacking both a hydrophobic antibiotic and a non-polar solvent (Table 1, last three rows) could not be coated due to poor drug solubility and too high viscosity. Hence, it was established that a hydrophobic antibiotic and/or a non-polar solvent has a viscosity-reducing effect.

A hydrophobic antibiotic (rifampicin, erythromycin, minocycline) could be dissolved in either a non-polar (chloroform) or polar (dimethyl sulfoxide, DMSO) solvent to yield a suitable coating solution (Table 1). Hence, the viscosity-reducing effect of a hydrophobic antibiotic may be irrespective of the solvent in which it is dissolved.

The sequence of mixing the polymer and antibiotic solutions was important to achieve a coating solution with low viscosity. I.e., mixing the hydrophobic antibiotic and/or a non-polar solvent with a polymer solution resulted in a low viscosity polymeric solution, which was maintained when subsequently mixed with a hydrophilic antibiotic solution (e.g., comprising tobramycin, gentamicin, vancomycin). In contrast, no viscosity-reducing effect was realized when first mixing the hydrophilic antibiotic and/or polar-solvent with a polymer solution. Hence, no low viscosity polymeric solution or low viscosity coating solution was yielded. Instead, this yielded a highly viscous solution when combined with a hydrophobic antibiotic solution, which could not be coated.

TABLE 1
Suitability of different coating solution formulations to form a thin-film coating on
a substrate, as determined by the drug solubility and viscosity of the solution. The
solution comprising antibiotic A in solvent A was first mixed with a solution comprising
PDLG in TFE. This mixture was then mixed with a solution comprising antibiotic B in
solvent B. Formulations comprising a hydrophobic antibiotic as first solvent and/or
a non-polar solvent (mixed with a drug-polymeric solution) showed excellent coatings
properties (groups 1-12), irrespective of the hydrophobic/hydrophilic drug combinations.
Formulations lacking a hydrophobic antibiotic as first solvent and a non-polar solvent
(mixed with a drug-polymeric solution) showed could not be coated.

| | Antibiotic A | | | | | | |
|---|---|---|---|---|---|---|---|
| | (hydrophilic or | | Antibiotic B | Solvent | | | |
| | hydrophobic) | Solvent A | (hydrophilic) | B | Solubility | Viscosity | Coatability |
| 1 | Rifampicin | Chloroform | Vancomycin | DMSO | High | Low | +++ |
| | (hydrophobic) | | | | | | |
| 2 | Erythromycin | Chloroform | Vancomycin | DMSO | High | Low | +++ |
| | (hydrophobic) | | | | | | |
| 3 | Minocycline | Chloroform | Vancomycin | DMSO | High | Low | +++ |
| | (hydrophobic) | | | | | | |
| 4 | Rifampicin | Chloroform | — | DMSO | High | Low | +++ |
| | (hydrophobic) | | | | | | |
| 5 | Erythromycin | Chloroform | — | DMSO | High | Low | +++ |
| | (hydrophobic) | | | | | | |
| 6 | Minocycline | Chloroform | — | DMSO | High | Low | +++ |
| | (hydrophobic) | | | | | | |
| 7 | Rifampicin | Chloroform | — | DMSO | High | Low | +++ |
| | (hydrophobic) + | | | | | | |
| | Erythromycin | | | | | | |
| | (hydrophobic) | | | | | | |
| 8 | Rifampicin | Chloroform | — | DMSO | High | Low | +++ |
| | (hydrophobic)+ | | | | | | |
| | Minocycline | | | | | | |
| | (hydrophobic) | | | | | | |
| 9 | Rifampicin | Chloroform | Tobramycin | DMSO | High | Low | +++ |
| | (hydrophobic) | | | | | | |
| 10 | Rifampicin | Chloroform | Gentamicin | DMSO | High | Low | +++ |
| | (hydrophobic) | | | | | | |
| 11 | — | Chloroform | Vancomycin | DMSO | High | Medium | + |
| 12 | Rifampicin | DMSO | Vancomycin | DMSO | High | High | +++ |
| | (hydrophobic) | | | | | | |
| 13 | — | — | Vancomycin | DMSO | Medium | High | − |
| 14 | — | DMSO | Vancomycin | DMSO | Low | High | − |
| 15 | Gentamicin | DMSO | Vancomycin | DMSO | Low | High | − |
| | (hydrophilic) | | | | | | |

3.2. Coatability of Antimicrobial Formulations with Different Polymers

All of the tested (hydrophobic) aliphatic polyesters (PDLG, PLC, PLDL, PCL, PL) were found to be suitable as carrier material for the current antimicrobial coating (Table 2). In contrast, the (hydrophilic) polyether-such as poly(ethylene glycol), did not provide a viscosity-reducing effect. Hence, the viscosity-reducing effect of hydrophobic antibiotic (rifampicin, erythromycin, minocycline) is particularly achieved when combining with a (hydrophobic) aliphatic polyester.

TABLE 2
Effect of hydrophilicity/polarity of the resorbable polymer in the manufacture of a coating
solution suitable to form a thin-film coating on a substrate, as determined by the drug
solubility and viscosity of the solution. Formulating comprising an aliphatic polyester
(mixed with a hydrophobic antibiotic and/or a non-polar solvent) showed excellent coatings
properties (groups 1-5). Formulations with a hydrophilic polyether mixed with a hydrophobic
antibiotic and/or a non-polar solvent) could not be coated (group 6). DMSO: dimethyl sulfoxide,
PDLG: DL-lactide/glycolide copolymer, PLC: L-lactide/caprolactone copolymer, PLDL:
L-lactide/DL-lactide copolymer, PCL: Polycaprolactone, PL: Poly(L-lactide).

| | | Antibiotic A | | Antibiotic B | | | | |
|---|---|---|---|---|---|---|---|---|
| | Polymer | (hydrophobic) | Solvent A | (hydrophilic) | Solvent B | Solubility | Viscosity | Coatability |
| 1 | PDLG | Rifampicin | Chloroform | Vancomycin | DMSO | High | Low | +++ |
| 2 | PCL | Rifampicin | Chloroform | Vancomycin | DMSO | High | Low | +++ |
| 3 | PL | Rifampicin | Chloroform | Vancomycin | DMSO | High | Low | +++ |
| 4 | PLDL | Rifampicin | Chloroform | — | DMSO | High | Low | +++ |
| 5 | PLC | Rifampicin | Chloroform | — | DMSO | High | Low | +++ |
| 6 | PEG | Rifampicin | Chloroform | Vancomycin | DMSO | Low | Low | − |

### 3.3. Drug Release Profiles

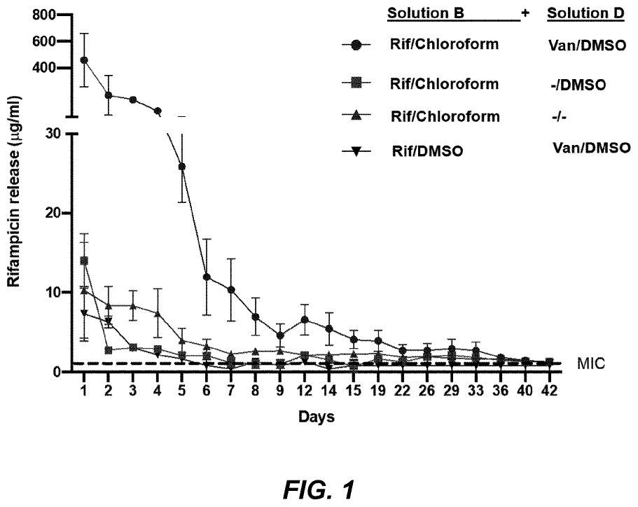

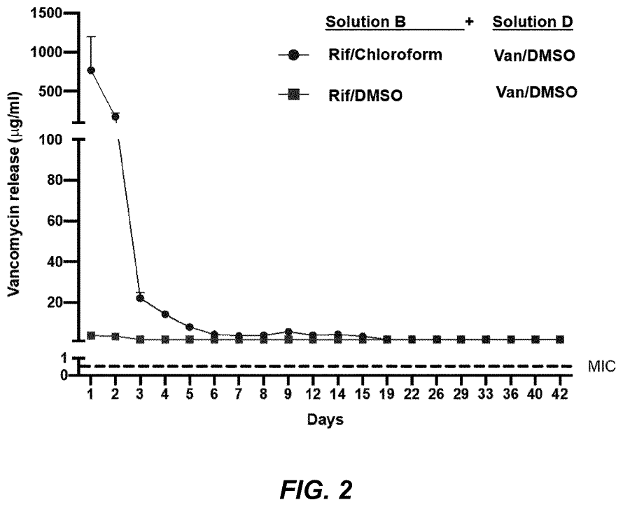

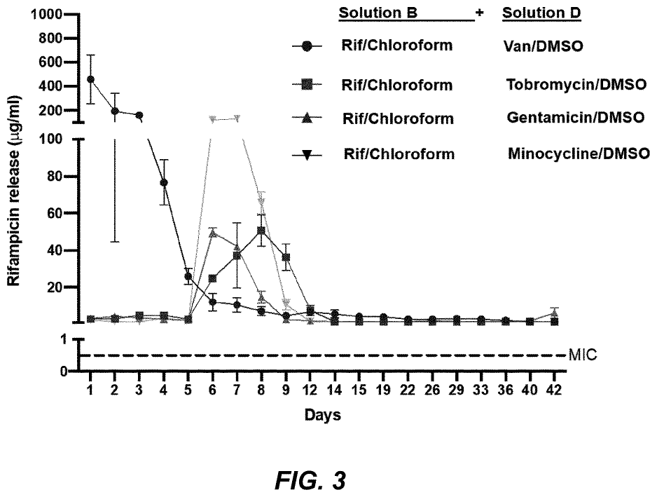

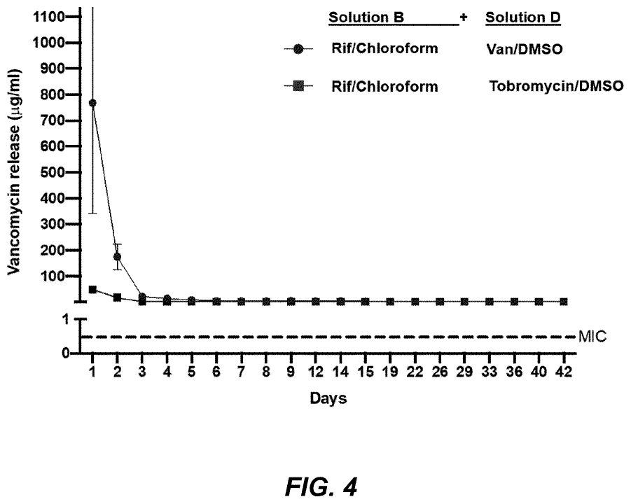

The drug release profiles demonstrated that the low viscosity coating solutions comprising at least a hydrophobic antibiotic (rifampicin) or a non-polar solvent (chloroform) yielded coatings with sustained release of rifampicin (FIG. 1) and vancomycin (FIG. 2). The coatings showed a release above the minimal inhibitory concentration (MIC) of Staphylococcus aureus up to six weeks. Similar sustained release profiles were achieved for different types of aliphatic polyester (co)polymers in the coating solution (FIG. 3, FIG. 4). This further supports that aliphatic polyester polymers may contribute to the viscosity-reducing effect and high drug loading efficiency.

3.4. Role of Viscosity in Coatability with Different Techniques

The viscosity of the antimicrobial formulation was found to influence the coating method that could be applied. It was found that electrospinning was most optimal at a viscosity of the antimicrobial formulation of (˜1-200 poise, 0.1-20 Pa·s). In this viscosity range, the initial jet may not break up into individual drops. Instead, the solvent may evaporate as the jet proceeds to the target, leaving behind a polymer fiber. It was found that at lower viscosities (e.g., 1-100 mPa·s), surface tension may be the dominant influence on fiber morphology and drops may form instead of fibers. Electrospraying was therefore possible in this viscosity range of 1-100 mPa·s of the antimicrobial formulation. It was found that spin coating may be performed for a relatively wide viscosity range.

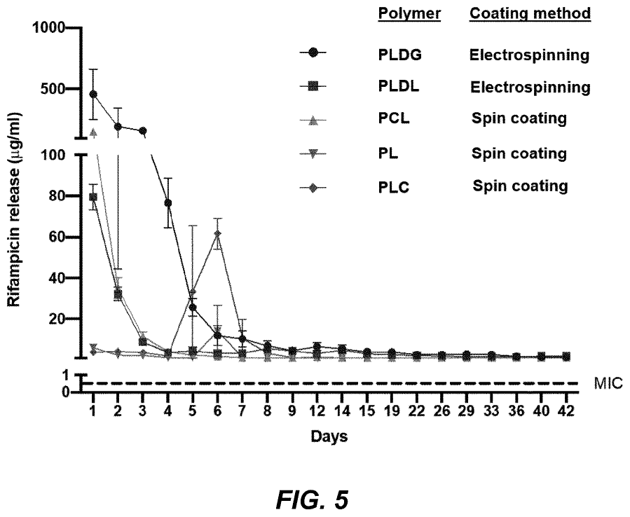

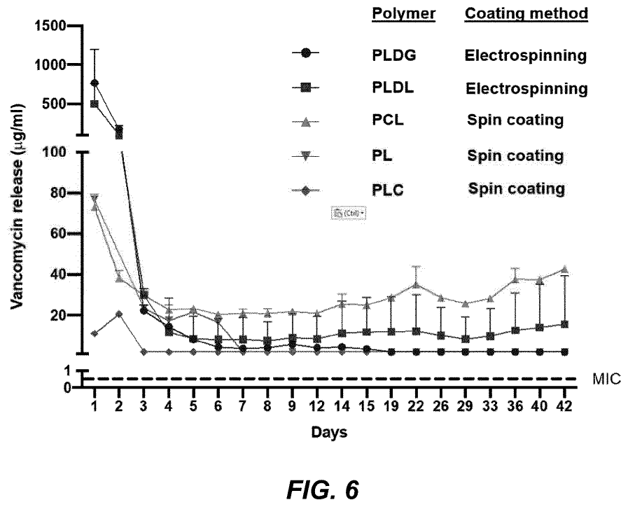

A sustained release profiles were seen for low viscosity coating solutions applied as coatings by means of different techniques, such as spraying (electrospraying), spin coating, or electrospinning (FIG. 5, FIG. 6). This strengthens that the viscosity-reducing effect of a hydrophobic antibiotic and/or a non-polar solvent allows use of different manufacturing techniques and may lead to several therapeutic applications.

### 3.5. Interaction Between Hydrophobic and Hydrophilic Antibiotic in Drug Release

It was found that the interaction between the hydrophilic and hydrophobic antibiotic in the coating may lead to their sustained drug release. The combination of rifampicin and vancomycin resulted into more sustained drug release, whereas a relatively higher burst release was seen for coatings comprising either rifampicin or vancomycin alone. The result is furthermore a comparable release profiles of both drugs, which is presumed to be more effective in eradicating infection.

Unexpectedly, it was found that the hydrophilic antibiotic (e.g., vancomycin) may enhance the diffusion of the hydrophobic antibiotic (e.g., rifampin) from the polymer matrix. Vice versa, the rifampicin may slow down the diffusion of vancomycin. The result is a comparable release profiles of both drugs, which is more effective in eradicating infection.

In this specific experiment, PLDG was dissolved to a concentration of 200 mg/ml (solution A), rifampicin was dissolved to a concentration of 50-70 mg/ml (solution B), and vancomycin was dissolved to a concentration of 50-70 mg/ml (solution D).

### 3.6. Antimicrobial Effect of the Developed Coatings

The long-term antimicrobial effect of coatings obtained with the low viscosity coating solution was shown in terms of the zone-of inhibition in inhibiting the growth of Staphylococcus aureus (the most common cause of metal implant infections). Antimicrobial activity was always seen after four and six weeks for coatings based on the low viscosity coating solution (Table 3), i.e., having a hydrophobic antibiotic and/or a non-polar solvent. The long-term antimicrobial activity of the coatings was observed irrespective of the type of aliphatic polyester used (Table 4). Furthermore, the long-term antimicrobial activity of the coatings was seen for all hydrophilic drug-hydrophobic drug combination tested (Table 5).

TABLE 2
Antimicrobial effect of coated implants against Staphyloccocus aureus.
Coatings manufactured using a coating solution comprising a
hydrophobic antibiotic as first solvent and a non-polar solvent
(mixed with a drug-polymeric solution) showed antimicrobial activity
for at least 6 weeks as shown by the zone of inhibition of bacterial
growth. PDLG (DL-lactide/glycolide copolymer) dissolved in TFE
(Tetrafluoroethylene) was used as polymeric solution in this experiment.
Coating was performed by electrospray.

| | |
|---|---|
| Antibiotic-solvent mixture | Zone of |

| | | |
|---|---|---|
| Hydrophobic/ | Hydrophilic/ | inhibition (cm) |

| solvent | solvent | Week 4 | Week 6 |
|---|---|---|---|
| —/ | —/ | 0 | 0 |
| chloroform | DMSO | | |
| Rifampicin/ | Vancomycin/ | 6.5 | 3.5 |
| chloroform | DMSO | | |
| —/ | Vancomycin/ | 0^{#} | 0^{#} |
| chloroform | DMSO | | |
| Rifampicin/ | Vancomycin/ | 7.0 | 3.0 |
| DMSO | DMSO | | |
| —/ | Vancomycin/ | 0^{#} | 0^{#} |
| DMSO | DMSO | | |
| Rifampicin/ | —/ | 6.5 | 2.5 |
| chloroform | DMSO | | |
| Rifampicin/ | —/— | 7.5 | 4.0 |
| chloroform | | | |

^{#}Electrosprayed coatings were non-uniform.

TABLE 4
Antimicrobial effect of coated implants against Staphyloccocus aureus.
Coatings manufactured using a coating solution comprising an
aliphatic polyester (dissolved in Tetrafluoroethylene) showed
antimicrobial activity for at least 6 weeks as shown by the zone
of inhibition of bacterial growth. The coating solution furthermore
comprised rifampicin/chloroform as first antibiotic solution
and vancomycin/dimethyl sulfoxide as second antibiotic solution.
Coating was performed by electrospray.

Zone of inhibition (cm)

| | Week 4 | Week 6 |
|---|---|---|
| PDLG | 7.5 | 3.5 |
| PCL | 8.0 | 3.5 |
| PL | 6.0 | 2.5 |
| PLDL | 5.5 | 2.5 |
| PLC | 6.0 | 2.5 |

PDLG: DL-lactide/glycolide copolymer, PLC: L-lactide/caprolactone copolymer, PLDL: L-lactide/DL-lactide copolymer, PCL: Polycaprolactone, PL: Poly(L-lactide).

TABLE 5
Antimicrobial effect of coated implants against Staphyloccocus aureus.
Coatings were manufactured using a polymeric solution based on
PDLG (DL-lactide/glycolide copolymer) dissolved in dissolved in
TFE (Tetrafluoroethylene) and different combinations of hydrophobic
(rifampicin, erythromycin, minocycline) antibiotics dissolved in
chloroform and hydrophilic antibiotics (vancomycin, tobramycin,
gentamycin) dissolved in dimethyl sulfoxide (DMSO). Antimicrobial
activity for at least 6 weeks was seen for the various combinations,
as shown by the zone of inhibition of bacterial growth.

| | |
|---|---|
| Antibiotic | Zone of inhibition (cm) |

| Hydrophobic | Hydrophilic | Week 4 | Week 6 |
|---|---|---|---|
| Rifampicin | Vancomycin | 7.0 | 4.0 |
| Erythromycin | Vancomycin | 2.5 | 1.5 |
| Rifampicin | Tobramycin | 6.5 | 3.5 |
| Rifampicin | Gentamycin | 6.5 | 3.5 |
| Rifampicin + | | 8.5 | 4.0 |
| Minocycline | | | |

It was found that the optimal drug:polymer ratio may be in the range of 1:1.5-1:2.5 (e.g., 1:2). For a higher drug amount relative to the polymer, the drugs showed a less sustained release profile. For a lower drug amount relative to the polymer, the drugs showed a more sustained release profile, however this was associated with a lower average drug elution per day (e.g., possibly below the required minimal inhibitory concentration).

### 4. Conclusion

The experiments identify a formulation for a low viscosity coating solution, which may form the basis for several coating strategies (e.g., spraying, spinning) and which have excellent sustained antimicrobial activity (at least six weeks demonstrated).

In particular, the coating obtainable with the low viscosity coating solution 1) is easily adaptable to incorporate different antibiotic of choice (preferably hydrophobic/non-polar and hydrophilic/polar drug combinations), 2) can be applied as a single polymer thin film coating, 3) can incorporate high drug content(s), and 4) can have sufficient low viscosity to be applied by different coating techniques, including electrospray.

Unexpectedly, it was found that a hydrophobic antibiotic (e.g., a rifamycin, a macrolide, a tetracylin) provided in a non-polar or a polar aprotic solvent may play an important role due to their viscosity-reducing effect in the coating solution. Moreover, the first antibiotic solution (i.e., preferably comprising a hydrophobic/non-polar antibiotic) is preferably first mixed with a non-polar organic solvent to achieve the largest viscosity-reducing effect. It was shown that the polymer is preferably an aliphatic polyester. Preferably the polymer is a hydrophobic aliphatic polyester, although it is considered that a hydrophilic aliphatic polyester may also work in certain embodiments.

It was found that the coating solution allows the coatings to be easily adaptable to multiple antibiotics of choice, even when the desirable antibiotics have distinct physicochemical properties (e.g., hydrophilicity/polarity). Lower viscosities may particularly allow coating by spraying, including electrospraying (e.g., at dynamic viscosity of 1 mPa·s-200 mPa·s) or by spinning, including spin coating and electrospinning (e.g., at dynamic viscosity of 200 mPa·s-20 Pa·s).

Surprisingly, the established coatings may show a beneficial interaction between incorporated (hydrophilic and hydrophobic drugs), leading to more sustained release and antimicrobial activity. Consequently, the established coatings were effective in eradicating Staphylococcus aureus (i.e., the most common cause of metal implant infections) over a wide time span.

### Example 2

### 1. Aim

The aim is to compare two protocols with respect to the properties of the coating solution and the coating that is formed thereby.

### 2. Methods

### Preparation of Coating Solution—Method A

1. 200 mg/mL of PDLG 5010 (Corbion) was dissolved in TFE (ABCR) in a glass container (20×15 mm) using a magnetic stirrer at 200 RPM overnight at room temperature and in darkness. The cap was sealed with parafilm. (Solution A)
2. 175 mg/mL of Rifampicin (USP, Cepham Life Sciences) was dissolved in chloroform (Sigma-Aldrich) in a 2 mL Eppendorf and mixed for 5 minutes using a vortex mixer. (solution B)
3. Solution B was added dropwise to solution A with a volume ratio of 70:30 (Solution A:B) by using a positive displacement pipette while mixing solution A with a magnetic stirrer. The mixture was allowed to mix for 2 hour in darkness. (solution C)
4. 250 mg/mL Vancomycin HCL (USP, Cepham Life Sciences) was dissolved in DMSO (Sigma-Aldrich) using a 2 mL Eppendorf and heating to 40° C. and mixing at 2,200 RPM using an Eppendorf Thermomixer mixer for 10 minutes. (Solution D)
4. Solution D was added dropwise to solution C with a volume ratio of 85:15 (solution C:D) by using a positive displacement pipette while mixing solution C with a magnetic stirrer at 1,500 RPM. The mixture was allowed to mix at room temperature at 1,500 RPM overnight for 24 hours in darkness. (solution E)

### Preparation of Coating Solution—Method B

1. 200 mg/mL of PCL (Corbion) was dissolved in Hexafluoroisopropanol (HFIP, Sigma-Aldrich) in a glass container (20×15 mm) using a magnetic stirrer at 200 RPM overnight at room temperature and in darkness. The cap was sealed with parafilm. 10 mg/mL of Rifampicin (USP, Cepham Life Sciences) was added directly to the PCL/HFIP solution. A shake table at 500 RPM and darkness was used to mix until a clear solution was observed (20 minutes). (solution A)
3. 10 mg/mL Vancomycin HCL (USP, Cepham Life Sciences) was dissolved in MilliQ water using a 2 mL Eppendorf and using a vortex mixer for 10 seconds until all solids were dissolved. The mixture was allowed to rest for 15 minutes in darkness. (Solution B)
4. Solution B was added dropwise to solution A with a volume ratio of 90:10 (Solution A:B) by using a pipette and while mixing solution A with a magnetic stirrer. The mixture was allowed to mix for 1 hour in presence of light. (solution C)

### Electrospray

The solutions were electrosprayed on titanium discs using the protocol described in Example 1 (section 2.3), using a needle of 22 G, voltage of 17 kV, distance of 8 cm, and a flowrate of 0.5-0.6 mL/h.

### Viscosity Measurement

The solutions according to Method A and Method B were placed on a shaker and the speed of the shaker was increased from 0 to 500 rpm and then decreased until a complete stop. The images were taken every second for 45 seconds. The horizontal lines indicate the flow of the solutions during the shaking process. The dynamic viscosity of the antimicrobial formulations was measured using the m-VROC viscometer (RheoSense).

### Coating Morphology

To characterize the coating morphology, atomic force microscopy (AFM, Ambios tech, US) was employed with a Nanosensors probe (silicon cantilever) under tapping mode in an air atmosphere. For each matrix, a scan of 20×20 mm was made for a precise test (n=5). The average roughness (Ra) was measured using Scanning probe image processor (SPIP) software (version 6.7, Image Metrology A/S, Denmark). The porosity and pore size was determined based on scanning electron microscopy images.

### 3. Results

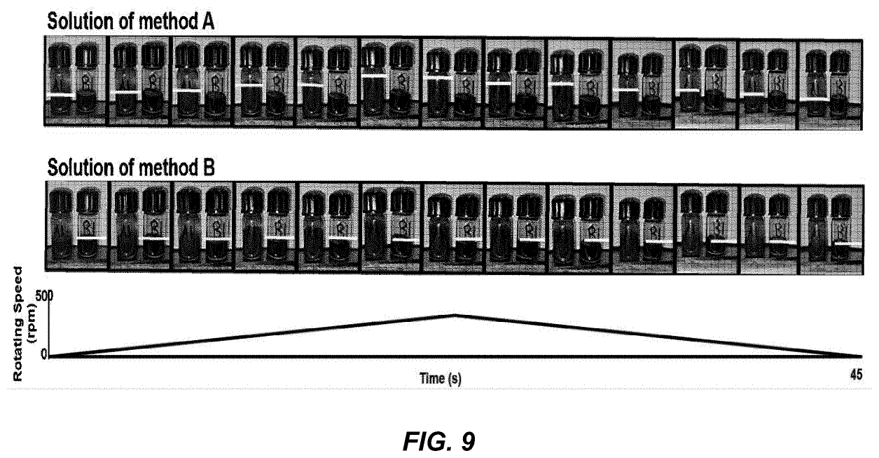

FIG. 9 shows that the coating solution obtained according to Method A has a higher flowability and lower viscosity as compared to the coating solution obtained according to Method B.

The coating solution according to Method A had a dynamic viscosity below 200 mPa·s, whereas the coating solution according to Method B had a larger dynamic viscosity (up the Pa·s range).

Using Method A, excellent drug solubility and miscibility, without drug precipitation.

Method B required more extensive stirring to avoid drug precipitation, indicating suboptimal drug solubility and miscibility.

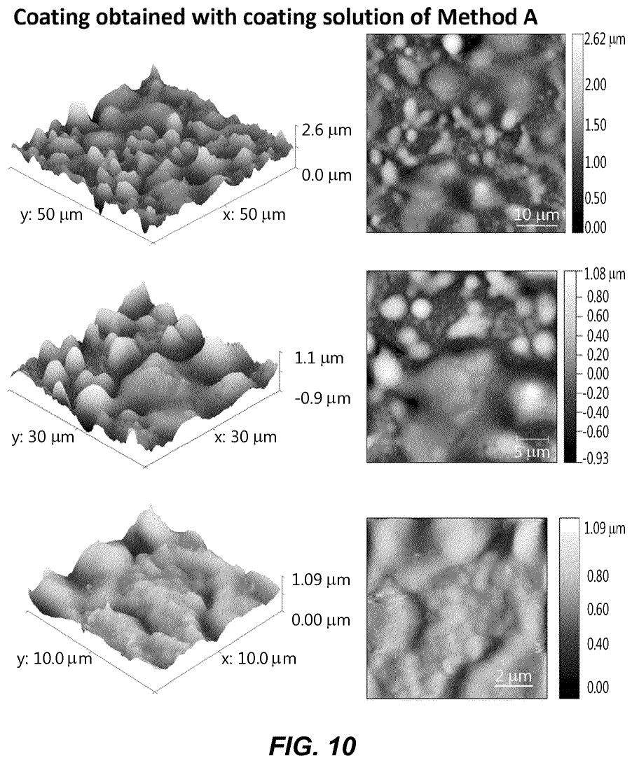

For Method A, the solution broke into droplets during electrospraying so that particles were deposited that combined into a homogeneous low-roughness film. This led to a dense and smooth coating (FIG. 10). The coating obtained according to Method A had no apparent pores.

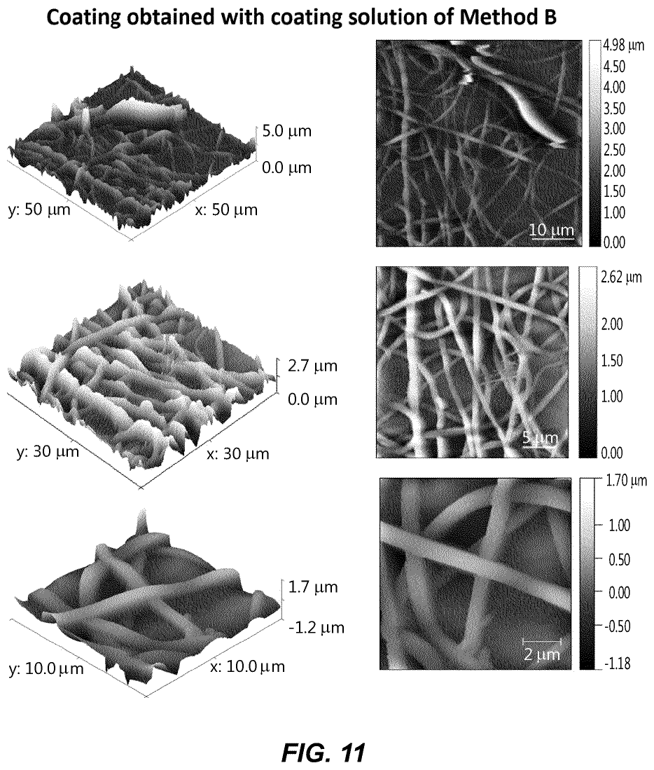

For Method B, the jet was kept in a continuous form to produce fibers with random orientation during electrospraying, instead of breaking into droplets. This led to a porous coating with higher surface roughness (FIG. 11). The coating obtained according to Method B had a porosity of 70-85% and average pore diameter of 1-10 μm.

The pore morphology of the fiber-based coating obtained by method B is in line with electrospun coatings reported literature (Biomaterials. 2008 May; 29(13):1989-2006; Adv Drug Deliv Rev. 2011 Apr. 30; 63(4-5):209-20).

Similar coatings are obtained when vancomycin is replaced with tobramycin, indicating that different hydrophilic antibiotics can be used. Similar results are obtained when using other hydrophobic aliphatic polyesters. For instance, a similar outcome is achieved when PLGA is used in Method B instead of PCL. It is also found that the fluorocarbon solvent is exchangeable. For instance, a similar outcome is achieved when hexafluoroisopropanol (HFIP) is used in Method A instead of tetrafluoroethylene (TFE).

The results of Example 2 are summarized in Table 6.

TABLE 6

| | Method A | Method |
|---|---|---|
| | (invention) | B (D2) |
| Antimicrobial formulation | | |
| Flowability | Excellent | Suboptimal |
| Dynamic viscosity | <200 mPa · s, | >200 mPa · s |
| Drug solubility | Excellent | Suboptimal |
| Drug precipitation | No | Yes |
| Antimicrobial coating | | |
| Droplet formation | Yes | No |
| Porosity (%) | Low/absent | 70-85% |
| Pore diameter (μm) | | 1-10 μm |
| Thin film coating | Yes | No |

1.-15. (canceled)

16. A method for preparing an antimicrobial formulation, the method comprising the following steps:
a) providing a first solvent with a polymer, wherein the first solvent is a fluorinated solvent and the polymer is an aliphatic polyester;
b) providing a second solvent with a first antibiotic, wherein the first antibiotic is hydrophobic and/or is soluble in a non-polar organic solvent;
c) mixing the first solvent with the polymer of step a) and the second solvent with the first antibiotic of step b) to obtain a mixture that has a lower dynamic viscosity than the first solvent with the polymer of step a);
d) providing a third solvent with a second antibiotic, wherein the second antibiotic is hydrophilic and/or soluble in a polar aprotic organic solvent; and
e) mixing the mixture obtained in step c) and the third solvent with the second antibiotic of step d) to obtain the antimicrobial formulation.

17. The method according to claim 16, wherein the mixture of step c) and/or the antimicrobial formulation has a dynamic viscosity of 1 mPa·s-20 Pa·s (pascal-second).

18. The method according to claim 16, wherein the aliphatic polyester comprises a hydrophobic aliphatic polyester.

19. The method according to claim 18, wherein the hydrophobic aliphatic polyester is at least one of DL-lactide/glycolide copolymer (PDLG), L-lactide/glycolide copolymer (PLGA), L-lactide/caprolactone copolymer (PLC), polycaprolactone (PCL), poly(D-lactide (PD), PL [poly(L-lactide)], or (poly(DL-lactide) (PDL).

20. The method according to claim 16, wherein the first antibiotic is at least one of a rifamycin, rifampin, a hydrophobic hydroquinolone, a macrolide, a tetracycline, and a lincosamide.

21. The method according to claim 16, wherein the second antibiotic is at least one of a glycopeptide, an aminoglycoside, vancomycin, a beta-lactam, a carbapenem, a hydrophilic fluoroquinolone, and a streptogramin.

22. The method according to claim 16, wherein the fluorinated solvent is a fluorocarbon, a fluoroalcohol, tetrafluoroethylene, hexafluoroisopropanol, trifluoroethanol, trifluoropropanol, or a mixture thereof.

23. The method according to claim 16, wherein:
the second solvent is a non-polar organic solvent, a polar aprotic organic solvent, chloroform, pentane, hexane, benzene, diethyl ether, and 1,40-dioxane or a mixture thereof; and/or
the third solvent is at least one of a polar aprotic organic solvent, dimethyl sulfoxide, dichloromethane, tetrahydrofuran, ethyl acetate, acetonitrile, dimethylformamide, dimethyl sulfoxide, acetone, and hexamethylphosphoric triamide.

24. The method according to claim 16, wherein:
in step a) the polymer is present in the first solvent in a concentration of 50-500 mg/ml;
in step b) the first antibiotic is present in the second solvent in a concentration of 25-500 mg/ml;
the mixing in step c) is performed at a ratio between second solvent with first antibiotic and first solvent with polymer of 1:1-1:2.5;
in step d) the second antibiotic is present in the third solvent in a concentration of 25-500 mg/ml; and/or
in step e) the mixing is performed at a ratio between third solvent with second antibiotic and mixture of step c) of 1:2-1:7.

25. The method according to claim 16, further comprising: preparing an antimicrobial coating with the thus obtained antimicrobial formulation by dip coating, spin coating, spraying, electrospraying, electrospinning, electrophoretic deposition, sputter coating, thermal spraying, plasma spraying, sol-gel, or layer-by-layer coating.

26. An antimicrobial formulation comprising:
a polymer at a concentration of 10-300 mg/ml of the antimicrobial formulation, wherein the polymer is an aliphatic polyester, a hydrophobic aliphatic polyester, DL-lactide/glycolide copolymer (PDLG), L-lactide/glycolide copolymer (PLGA), L-lactide/caprolactone copolymer (PLC), polycaprolactone (PCL), poly(D-lactide) (PD), poly(L-lactide) (PL), or (poly(DL-lactide) (PDL);
a first antibiotic at a concentration of 10-200 mg/ml of the antimicrobial formulation, wherein the first antibiotic is hydrophobic and/or soluble in a non-polar organic solvent;
a second antibiotic at a concentration of 10-200 mg/ml of the antimicrobial formulation wherein the second antibiotic is hydrophilic and/or soluble in a polar aprotic organic solvent;
a non-polar organic solvent at a concentration of 10-50% (v/v) with respect to the antimicrobial formulation; and
a polar aprotic organic solvent at a concentration of 1-40% (v/v) with respect to the antimicrobial formulation,
wherein the antimicrobial formulation has a dynamic viscosity of 1 mPa·s-200 mPa·s (pascal-second).

27. An antimicrobial coating comprising:
a polymer in an amount of 40-90%, by weight of the antimicrobial coating, wherein the polymer is an aliphatic polyester or a hydrophobic aliphatic polyester;
a first antibiotic in an amount of 5-40%, by weight of the antimicrobial coating, wherein the first antibiotic is hydrophobic and/or soluble in a non-polar organic solvent; and
a second antibiotic in an amount of 5-40%, by weight of the antimicrobial coating,
wherein the second antibiotic is hydrophilic and/or soluble in a polar aprotic organic solvent,
wherein the first antibiotic and the second antibiotic are comprised in a matrix effected by the polymer, and
wherein the antimicrobial coating has a porosity of no more than 10%.

28. The antimicrobial coating of claim 27, wherein:
the aliphatic polyester is at least one of DL-lactide/glycolide copolymer (PDLG), L-lactide/glycolide copolymer (PLGA), L-lactide/caprolactone copolymer (PLC), polycaprolactone (PCL), poly(D-lactide) (PD), poly(L-lactide) (PL), or (poly(DL-lactide) (PDL);
the first antibiotic is at least one of a rifamycin, a hydrophobic hydroquinolones, a macrolide, a tetracycline, rifampin, or a lincosamide; and/or
the second antibiotic is at least one of a glycopeptide, an aminoglycoside, vancomycin, a beta-lactam, a carbapenem, a hydrophilic fluoroquinolone, or a streptogramin.

29. The antimicrobial coating of claim 27, wherein:
the antimicrobial coating is a single layer antimicrobial coating;
the total polymer content in the antimicrobial coating is effected for 90-100 wt. % by a single polymer;
the surface of the antimicrobial coating is hydrophobic, as defined by a static water contact angle of 70 or more; and/or
the antimicrobial coating has an average surface roughness of 20-1000 nm.

30. A method of using the antimicrobial formulation of claim 26, the method comprising:
coating at least a portion of an orthopedic implant with the antimicrobial formulation.

31. The antimicrobial formulation of claim 26, wherein the first antibiotic is selected from the group consisting of a rifamycin, rifampin, a hydrophobic hydroquinolone, a macrolide, a tetracycline, a lincosamide, and a mixture of any thereof and is at a concentration of 20-100 mg/ml of the antimicrobial formulation.

32. The antimicrobial formulation of claim 26, wherein the second antibiotic is selected from the group consisting of a glycopeptide, an aminoglycoside, vancomycin. a beta-lactam, a carbapenem, a hydrophilic fluoroquinolone, a streptogramin, and a mixture of any thereof and is at a concentration of 20-100 mg/ml of the antimicrobial formulation.

33. The antimicrobial formulation of claim 26, wherein the non-polar organic solvent is selected from the group consisting of chloroform, pentane, hexane, benzene, diethyl ether, and 1,40-Dioxane, and a mixture of any thereof and is at a concentration of 20-40% (v/v) with respect to the antimicrobial formulation.

34. The antimicrobial formulation of claim 26, wherein the polar organic solvent is selected from the group consisting of dimethyl sulfoxide, dichloromethane, tetrahydrofuran, ethyl acetate, acetonitrile, dimethylformamide, dimethyl sulfoxide, acetone, and hexamethylphosphoric triamide, and a mixture of any thereof and is at a concentration of 10-30% (v/v) with respect to the antimicrobial formulation.
